(19) United States
(12) Patent Application Publication
(10) Pub. No.: US 2024/0042153 A1
(43) Pub. Date: Feb. 8, 2024

(54) MASK

(71) Applicant: Fisher & Paykel Healthcare Limited, Auckland (NZ)

(72) Inventors: Kevin Blake POWELL, Auckland (NZ); Jason Allan KLENNER, Auckland (NZ); Kevin Peter O'DONNELL, Auckland (NZ); Monika BAUMANN, Auckland (NZ); Rory Alexander MONRO, Auckland (NZ); Callum James Thomas SPENCE, Auckland (NZ); Michael Paul RONAYNE, Auckland (NZ)

(21) Appl. No.: 17/999,426

(22) PCT Filed: May 27, 2021
(86) PCT No.: PCT/IB2021/054620

§ 371 Date: Nov. 21, 2022

Related U.S. Application Data
(60) Provisional application No. 63/056,434, filed on Jul. 24, 2020.
(60) Provisional application No. 63/031,167, filed on May 28, 2020.

(51) Int. Cl. A61M 16/06; A61M 16/10

(52) U.S. Cl. CPC A61M 16/0622; A61M 16/0666; A61M 16/1065

(57) ABSTRACT

A mask 100 configured to substantially surround an opening of a patient's airway. The mask has a mask body 101 comprising a filter 103 configured to filter a fluid from a patient facing side of the mask to a non-patient facing side 105 of the mask, wherein the non-patient facing side faces an ambient environment AE. The mask has an interfacing feature 121 configured to, in use, interface with a patient interface 10100, 10100′ provided on the patient 10020.

### TECHNICAL FIELD

The present disclosure relates to masks for use with respiratory assistance systems.

### BACKGROUND

During application of high-flow therapy with a non-sealing patient interface, gases exhaled from a patient may travel a significant distance depending on the therapy flow rate. When a patient coughs, sneezes and/or breathes heavily, this distance may be further increased. This exhaled air may contain contaminants and aerosols that are undesirable for other persons who may be in proximity to the patient. The aerosols may, for example, contain pathogens or nebulised drugs (which may have undesirable effects on the other persons).

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY

In accordance with certain features, aspects and advantages of at least one of the embodiments disclosed herein, a mask configured to substantially surround an opening of a patient's airway is provided, the mask comprising: a mask body comprising a filter configured to filter a fluid from a patient facing side of the mask to a non-patient facing side of the mask, wherein the non-patient facing side faces an ambient environment; and an interfacing feature configured to, in use, interface with a patient interface provided on the patient.

In some configurations, the filter is configured to limit travel of a liquid from the patient-facing side of the mask.

In some configurations, the filter comprises a material that is permeable to water molecules and gases flow.

In some configurations, the filter comprises a material that is substantially impermeable to bulk flow of liquid water.

In some configurations, the mask comprises an absorbent material to absorb the liquid.

In some configurations, the filter comprises the absorbent material.

In some configurations, the absorbent material is hydrophilic, optionally the absorbent material comprises a hydrophilic material or comprises a material that has been treated to be hydrophilic.

In some configurations, the absorbent material is configured to wick the liquid away from the patient.

In some configurations, the absorbent material is configured to permit evaporation of the liquid away from the patient, optionally to the ambient environment.

In some configurations, the mask comprises a transmission arrangement configured to transfer the liquid absorbed by the absorbent material away from the absorbent material.

In some configurations, the transmission arrangement is configured to transfer the absorbed liquid to an absorbent pad.

In some configurations, the absorbent material comprises one or more of polyester, polyurethane, nylon, polyethylene or a composite thereof.

In some configurations, the filter comprises a fabric, optionally a textile fabric and/or a polymer matrix fabric.

In some configurations, the mask body and/or filter is elastic or comprises an elastic material.

In some configurations, the mask body and/or filter is/are configured to minimize impediment on flow rates of a gases flow through the mask body and/or filter.

In some configurations, the mask body and/or filter is configured to allow a flow rate of a gases flow through it of up to about 450 L/min.

In some configurations, the mask body and/or filter is configured to allow a flow rate of a gases flow through the mask body and/or filter of about 10-450 L/min.

In some configurations, the filter is porous.

In some configurations, the mask comprises a moisture indicator configured to provide an indication of the moisture in the filter.

In some configurations, the moisture indicator comprises a hydrochromatic material or coating that changes colour when a predetermined moisture level is reached.

In some configurations, the filter is removable from the mask.

In some configurations, the patient interface comprises a non-sealing patient interface.

In some configurations, the patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s).

In some configurations, the mask is adapted to cover a nose and mouth of the patient.

In some configurations, the mask is selectively securable to or removable from the patient independently of the nasal cannula.

In some configurations, the mask does not comprise a gases supply or gases removal conduit and/or does not comprise a connection port for connecting to a gas source or gas removal device and/or is not adapted to actively deliver a flow of gases to the patient and/or is not adapted to actively remove a flow of gases from the patient.

In some configurations, the mask comprises a gases removal port or conduit to transmit fluids from the patient facing side of the mask to a remote filter or gases removal device.

In some configurations, the mask comprises a frame, wherein the mask body is integral with or releasably coupled to the frame.

In some configurations, the frame is sewn, heat welded, ultrasonically welded or overmolded to the mask body.

In some configurations, the frame comprises a pre-formed shape that conforms to the shape of the patient's face.

In some configurations, the frame is rigid or more rigid than the mask body and/or filter.

In some configurations, the mask comprises headgear connection features, optionally on the frame and/or mask body.

In some configurations, the mask comprises a headgear, optionally that is integral with the mask or is releasably connectable to the headgear connection features.

In some configurations, the mask comprises a pair of upper headgear connection features and a pair of lower headgear connection features.

In some configurations, the headgear comprises an upper headstrap connectable to the upper headgear connection features and a lower headstrap connectable to the lower headgear connection features, wherein the upper and lower headstraps are adapted to extend around a back of the patient's head in use.

In some configurations, the headgear comprises: a first side strap that is connectable to one of the two upper headgear connection features and connectable to one of the two lower headgear connection features; a second side strap that is connectable to the other of the two upper headgear connection features and connectable to other of the two lower headgear connection features; wherein the first and second side straps are configured to loop around the ears of the patient in use.

In some configurations, the headgear is adjustable.

In some configurations, the mask comprises a cushion configured to seal about or against the patient's face.

In some configurations, the patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s), and wherein the interfacing feature comprises a recess or slot in the cushion, wherein the recess or slot conforms to an external surface of the nasal cannula such that the cushion substantially seals about the side arms and/or patient conduit of the nasal cannula.

In some configurations, the recess comprises a key-hole shape recess.

In some configurations, the recess extends from a surface that contacts the patient's face in use to a portion of the cushion distal from the patient's face.

In some configurations, the patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s), wherein the interfacing feature comprises an aperture provided in the mask body to allow a portion of the nasal cannula to extend through, optionally to allow the patient conduit of the nasal cannula to extend through.

In some configurations, the aperture is provided in a side of the mask body.

In some configurations, the mask comprises two of said apertures, optionally in opposing sides of the mask body.

In some configurations, the mask body and/or filter comprises a pre-formed shape.

In some configurations, the mask body and/or filter is rigid or semi-rigid.

In some configurations, the filter is provided on an underlying material that imparts a pre-formed shape to the filter.

In some configurations, the mask body is substantially formed of the filter.

In some configurations, the mask body and/or the filter comprises a pre-formed shape that is configured to deflect fluids from the opening of a patient's airway in an intended direction.

In some configurations, the patient interface comprises one or more venting apertures, and the mask is configured to filter and/or deflect fluids that exit the patient interface via the venting aperture(s).

In some configurations, the mask body comprises a filter housing, wherein the filter is in the filter housing, and wherein optionally the filter comprises a pleated, corrugated, or concertina configuration filter.

In some configurations, the patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s), wherein the mask comprises a frame shaped to conform to an external surface of the patient conduit of the nasal cannula, and wherein the interfacing feature comprises a first mounting structure to releasably couple the frame to the patient conduit.

In some configurations, the first mounting structure comprises a frame mount adapted to engage with a tube clip lug of a tube clip of the patient conduit.

In some configurations, the interfacing feature comprises a second mounting structure to releasably couple the frame to a side arm of the nasal cannula.

In some configurations, the second mounting structure comprises a frame lug and at least one side arm of the nasal cannula comprises a side arm mount, the frame lug adapted to engage with the side arm mount.

In some configurations, the patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s), wherein the interfacing feature comprises at least one mounting mechanism to couple the mask to the nasal cannula.

In some configurations, an edge of the mask comprises an absorbent material.

In some configurations, the mask comprises an absorbent pad.

In some configurations, the mask body comprises a transparent portion to permit a view of a portion of the patient's face during use.

In some configurations, the transparent portion is arranged at an upper portion of the mask to permit a view of the patient's nose during use.

In some configurations, the mask comprises a deformable structure that can be shaped to follow a contour of the patient's face.

In some configurations, the deformable structure comprises a malleable structure, optionally a metal wire or metal strip.

In some configurations, the deformable structure is provided at a top portion of the mask body, optionally at or proximal to the patient's nasal bridge when in use.

In some configurations, the patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s), wherein the interfacing mechanism comprises an aperture through which a portion of the nasal cannula is adapted to extend, optionally wherein the interfacing mechanism comprises two apertures, optionally one on either side of the mask.

In some configurations, the mask comprises an attachment mechanism to releasably attach the mask to the patient's face.

In some configurations, the attachment mechanism comprises a first portion configured to adhere to the patient's face and a second portion provided on the mask, the first and second portions adapted to releasably couple with one another.

In some configurations, the attachment mechanism comprises a hook-and-loop fastener system.

In some configurations, the aperture(s) is/are provided on a tab or tabs extending from a side or sides of the mask.

In some configurations, the mask comprises a headgear with a strap coupled to a periphery of the mask body.

In some configurations, the headgear comprises a crownstrap.

In some configurations, an aperture is provided between the strap and the mask body, the aperture configured such that a portion of a nasal cannula can extend through the aperture.

In some configurations, the mask comprises a chin portion that extends under the patient's chin in use.

In some configurations, the chin portion is formed from the filter.

In some configurations, the chin portion comprises an elastic material.

In some configurations, the mask body is formed substantially entirely of the filter.

In some configurations, the mask is configured to extend around a back of the patient's head, and does not comprise a headgear.

In some configurations, the mask is configured to extend downwardly from the patient's face towards the patient's chest in use.

In some configurations, the mask comprises a non-sealing mask.

In some configurations, the patent interface comprises a non-sealing nasal cannula, and the mask body and/or filter comprises a flexible material configured to conform to the nasal cannula.

In some configurations, the mask body and/or filter is elastic or the flexible material is an elastic material.

In some configurations, the interfacing feature comprises a recess or slot that enables a portion of the nasal cannula to extend therethrough.

In some configurations, the recess or slot is configured to conform to the portion of the nasal cannula to substantially seal about the portion of the nasal cannula.

In some configurations, a portion of the mask body is configured to extend around and/or be coextensive with a patient conduit of the nasal cannula.

In accordance with certain features, aspects and advantages of at least one of the embodiments disclosed herein, an interface system is provided, the interface system comprising: a non-sealing patient interface configured to deliver a gases flow to a patient's airway; and a mask comprising: a mask body comprising a patient facing side and a non-patient facing side, the patient facing side configured to form an interior space with the patient's face when the mask is in contact with the patient, the interior space configured to accommodate a portion of the non-sealing patient interface; a filter configured to filter a fluid from a patient-facing side of the mask to a non-patient facing side of the mask, wherein the non-patient facing side faces an ambient environment; and an interfacing feature configured to, in use, interface with the non-sealing patient interface.

In some configurations, the non-sealing patient interface comprises a first retention mechanism configured to retain said patient interface on the patient's face in use.

In some configurations, the interface system comprises a second retention mechanism configured to retain the mask on the patient's face in use, wherein the first and second retention mechanisms are separate such that the mask is selectively securable to or removable from the patient independent of the non-sealing patient interface.

In some configurations, the mask does not comprise a gases supply or gases removal conduit and/or does not comprise a connection port for connecting to a gas source or gas removal device and/or is not adapted to actively deliver a flow of gases to the patient and/or is not adapted to actively remove a flow of gases from the patient.

In some configurations, the gases flow to the patient is only provided by the non-sealing patient interface.

In some configurations, the filter is configured to limit travel of a liquid from the patient-facing side of the mask.

In some configurations, the filter comprises a material that is permeable to water molecules and gases flow.

In some configurations, the filter comprises a material that is substantially impermeable to bulk flow of liquid water.

In some configurations, the mask comprises an absorbent material to absorb the liquid.

In some configurations, the filter comprises the absorbent material.

In some configurations, the absorbent material is hydrophilic, optionally wherein the absorbent material comprises a hydrophilic material or comprises a material that has been treated to be hydrophilic.

In some configurations, the absorbent material is configured to wick the liquid away from the patient.

In some configurations, the absorbent material is configured to permit evaporation of the liquid away from the patient, optionally to the ambient environment.

In some configurations, the mask comprises a transmission arrangement configured to transfer the liquid absorbed by the absorbent material away from the absorbent material.

In some configurations, the transmission arrangement is configured to transfer the absorbed liquid to an absorbent pad.

In some configurations, the absorbent material comprises one or more of polyester, polyurethane, nylon, polyethylene or a composite thereof.

In some configurations, the filter comprises a fabric, optionally a textile fabric and/or a polymer matrix fabric.

In some configurations, the mask body and/or filter is elastic or comprises an elastic material.

In some configurations, the mask body and/or filter configured to minimize impediment on flow rates of a gases flow through the mask body and/or filter.

In some configurations, the mask body and/or filter is configured to allow a flow rate of a gases flow through it of up to about 450 L/min.

In some configurations, the mask body and/or filter is configured to allow a flow rate of a gases flow through the mask body and/or filter of about 10-450 L/min.

In some configurations, the non-sealing patient interface comprises a nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s).

In some configurations, the mask comprises a cushion configured to seal about or against the patient's face, and wherein the interfacing feature comprises a recess or slot in the cushion, wherein the recess conforms to an external surface of the nasal cannula such that the cushion substantially seals about the side arms and/or patient conduit of the nasal cannula.

In some configurations, the recess comprises a key-hole shape recess.

In some configurations, the recess extends from a surface that contacts the patient's face in use to a portion of the cushion distal from the patient's face.

In some configurations, the interfacing feature comprises an aperture provided in the mask body and/or between the mask body and the second retention mechanism to allow a portion of the nasal cannula to extend through, optionally to allow the patient conduit of the nasal cannula to extend through.

In some configurations, the mask comprises a frame shaped to conform to an external surface of the patient conduit of the nasal cannula, and wherein the interfacing feature comprises a first mounting structure to releasably couple the frame to the patient conduit.

In some configurations, the first mounting structure comprises a frame mount adapted to engage with a tube clip lug of a tube clip of the patient conduit.

In some configurations, the interfacing feature comprises a second mounting structure to releasably couple the frame to a side arm of the nasal cannula.

In some configurations, the second mounting mechanism comprises a mask frame lug and at least one side arm of the comprises a side arm mount, the frame lug adapted to engage with the side arm mount.

In accordance with certain features, aspects and advantages of at least one of the embodiments disclosed herein, a mask configured to substantially surround an opening of a patient's airway is provided, the mask comprising: a mask body comprising a filter configured to enable a flow of gases from a patient facing side of the mask to a non-patient facing side of the mask in use and configured to limit travel of a liquid from the patient facing side of the mask to a the non-patient facing side of the mask and capture the liquid, and a transmission arrangement to transfer the liquid from the filter to a reservoir.

In some configurations, the filter comprises a material that is permeable to water molecules and gases flow.

In some configurations, the filter comprises a material that is substantially impermeable to bulk flow of liquid water.

In some configurations, the mask comprises an absorbent material to absorb the liquid.

In some configurations, the absorbent material is hydrophilic, optionally wherein the absorbent material comprises a hydrophilic material or comprises a material that has been treated to be hydrophilic.

In some configurations, the absorbent material is configured to wick the liquid away from the patient.

In some configurations, the absorbent material is configured to permit evaporation of the liquid away from the patient, optionally to an ambient environment.

In some configurations, the reservoir comprises an absorbent pad.

In some configurations, the absorbent pad is arranged to be positioned under or proximal to the patient's chin during use.

In some configurations, the transmission arrangement is integral with the filter.

In some configurations, the filter comprises a first absorbent material and the transmission arrangement comprises a second absorbent material.

In some configurations, the first absorbent material and the second absorbent material are the same.

In some configurations, the first absorbent material and the second absorbent material are different or have different properties.

In some configurations, the second absorbent material is more absorbent than the first absorbent material.

In some configurations, the second absorbent material comprises a thickness greater than a thickness of the first absorbent material.

In some configurations, the second absorbent material is shaped to facilitate transfer and movement of the liquid from the filter to the reservoir.

In some configurations, the absorbent material(s) and/or absorbent pad comprises one or more of a polyester, polyurethane, nylon, polyethylene or composite thereof.

In some configurations, the filter comprises a fabric, optionally a textile fabric and/or a polymer matrix fabric.

In some configurations, the mask body and/or filter is elastic or comprises an elastic material.

In some configurations, the mask body and/or filter is configured to minimize impediment on flow rates of a gases flow through the mask body and/or filter.

In some configurations, the mask body and/or filter is configured to allow a flow rate of a gases flow through it of up to about 450 L/min.

In some configurations, the mask body and/or filter is configured to allow a flow rate of a gases flow through the mask body and/or filter of about 10-450 L/min.

In some configurations, the filter is porous.

In some configurations, the mask comprises a moisture indicator configured to provide an indication of the moisture in the filter.

In some configurations, the moisture indicator comprises a hydrochromatic material or coating that changes colour when a predetermined moisture level is reached.

In some configurations, the moisture indicator is on a front, non-patient surface of the mask body.

In some configurations, the mask comprises an upper portion adapted to cover the patient's nose and/or mouth in use, and a lower portion adapted to position under the patient's chin in use.

In some configurations, the lower portion comprises discontinuous regions that are configured to overlap and couple with one another to form a chin strap.

In some configurations, the discontinuous regions comprise a fastening mechanism to couple said discontinuous regions together.

In some configurations, the fastening mechanism comprises a hook-and-loop fastener system.

In some configurations, the mask comprises a headgear that is integral to or releasably connectable to the mask body.

In accordance with certain features, aspects and advantages of at least one of the embodiments disclosed herein, a mask configured to substantially surround an opening of a patient's airway is provided, the mask comprising: a mask body configured to define a cavity or part thereof with the patient's face in use, the cavity configured to at least accommodate a portion of the opening of the patient's airway, the mask body configured to enable a flow of gases from a patient facing side of the mask to a non-patient facing side of the mask in use, the mask body comprising a transparent portion to permit a view of a portion of the patient's face in use; and an absorbent material configured to absorb moisture within the cavity during use.

In some configurations, the cavity is adapted to accommodate the patient's nose and mouth in use.

In some configurations, the transparent portion is arranged at an upper portion of the mask to permit a view of the patient's nose during use.

In some configurations, the transparent portion is hydrophobic, comprises a hydrophobic material or is treated to be hydrophobic.

In some configurations, the mask comprises a filter configured to filter a fluid from a patient facing side of the mask to a non-patient facing side of the mask.

In some configurations, the filter is configured to limit travel of a liquid from the patient-facing side of the mask.

In some configurations, the filter comprises the absorbent material.

In some configurations, the absorbent material extends from the filter to a portion of the mask body.

In some configurations, the filter is arranged at a lower portion of the mask.

In some configurations, the filter is arranged to be proximal the patient's mouth in use.

In some configurations, the mask comprises a moisture indicator configured to provide an indication of the moisture in the filter and/or the absorbent material.

In some configurations, the moisture indicator comprises a hydrochromatic material or coating that changes colour when a predetermined moisture level is reached.

In some configurations, the filter is removable from the mask.

In some configurations, the absorbent material is hydrophilic, optionally wherein the absorbent material comprises a hydrophilic material or comprises a material that has been treated to be hydrophilic.

In some configurations, the mask comprises a seal that is configured to seal about or against the patient's face.

In some configurations, the mask body is shaped to conform to the patient's face.

In some configurations, one or more of the absorbent material, filter, and mask body is/are formed of or comprise(s) a material that is permeable to water molecules and gases flow and/or hydrophilic material.

In some configurations, one or more of the absorbent material, filter, and mask body is/are formed of or comprise(s) a material that is substantially impermeable to bulk flow of liquid water.

In some configurations, the mask comprises a headgear, wherein the headgear comprises an upper headstrap having ends that connect to an upper portion of the mask body, and wherein the headgear comprises a lower headstrap having ends that connect to a lower portion of the mask body.

In some configurations, the mask does not comprise a gases supply or gases removal conduit and/or does not comprise a connection port for connecting to a gas source or gas removal device and/or is not adapted to actively deliver a flow of gases to the patient and/or is not adapted to actively remove a flow of gases from the patient.

In some configurations, the mask body comprises a frame, and

wherein a remainder of the mask body is integral with or releasably coupled to the frame.

In some configurations, the frame is sewn, heat welded, ultrasonically welded or overmolded to the mask body.

In some configurations, the frame comprises a pre-formed shape that conforms to the shape of the patient's face.

In some configurations, the frame is rigid or more rigid than the mask body and/or filter.

In some configurations, the frame is configured to extend across the patient's eyes in use.

In some configurations, the frame comprises a transparent material that provides the transparent portion of the mask body.

In some configurations, a portion of the frame comprises a material that dispels liquids or transmits liquids away from said portion or wherein said portion of the frame is treated to dispel liquid or transmit liquid away from said portion.

In some configurations, the frame is configured to extend towards the back of the patient's ears to retain the mask on the patient's face in use.

In some configurations, the frame comprises a first material extending along a length of the frame and arranged to be positioned below the patient's eyes in use.

In some configurations, the first material comprises the absorbent material.

In some configurations, the frame comprises a second material extending along a length of the frame and arranged to be positioned above the patient's eyes in use.

In some configurations, the first material and the second material are configured to seal about or against the patient's face.

In some configurations, the first material and the second material form a periphery around a portion of the patient's face in use, optionally around at least the patient's eyes.

In some configurations, the mask body is removably attached to the frame, optionally via a hook-and-loop fastener system.

In some configurations, the mask body comprises an aperture to provide for a portion of a patient interface to extend through when in use, when the mask is placed over the patient interface.

In some configurations, the second patient interface comprises a non-sealing nasal cannula.

In some configurations, an upper portion of the mask is configured to couple with a headgear that retains the mask on the patient.

In some configurations, the headgear comprise a cap.

In accordance with certain features, aspects and advantages of at least one of the embodiments disclosed herein, an interface system is provided, the interface system comprising: a non-sealing patient interface configured to deliver a gases flow to a patient's airway; and the mask as outlined above.

In some configurations, the mask comprises an interfacing feature to, in use, interface with the non-sealing patient interface.

In some configurations, the interfacing feature comprises a portion of the seal being more compliant than other portions of the seal, wherein the compliant portion is configured to conform to an external portion of the non-sealing patient interface during use.

In some configurations, the interfacing feature comprises an aperture or recess in the mask body, optionally wherein the recess comprises a slot.

In some configurations, the aperture or recess is arranged in a side of the mask body.

In some configurations, the aperture comprises an elastomer with a tearable portion to allow a portion of the non-sealing patient interface to extend therethrough during use.

In some configurations, the mask comprises an at least partly removable cover that is configured to cover the aperture in a first configuration and to expose the aperture in a second configuration.

In some configurations, the at least partly removable cover is adapted to couple with a portion of the mask body via a fastening mechanism, optionally via a hook-and-loop fastener system.

In some configurations, the interfacing feature comprises a recess or slot in the mask body, and wherein an end of the slot is covered by a portion of a mask headstrap to define an aperture to allow a portion of the non-sealing patient interface to extend through the aperture during use.

In some configurations, the interfacing feature comprises a recess or slot in the mask body, and comprising a coupling mechanism to couple ends of the recess or slot together to define an aperture to allow a portion of the non-sealing patient interface to extend through the aperture during use.

In some configurations, the non-sealing patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s).

In some configurations, the non-sealing nasal cannula comprises a cannula headgear configured to retain said non-sealing nasal cannula on the patient's face in use.

In some configurations, the mask comprises a mask headgear, wherein the cannula headgear and mask headgear are separate such that the mask is selectively securable to or removable from the patient independently of the non-sealing nasal cannula.

In some configurations, the mask does not comprise a gases supply or gases removal conduit and/or does not comprise a connection port for connecting to a gas source or gas removal device and/or is not adapted to actively deliver a flow of gases to the patient and/or is not adapted to actively remove a flow of gases from the patient.

In some configurations, the gases flow to the patient is only provided by the nasal cannula.

In some configurations, the non-sealing patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s), and wherein the gases flow provided by the nasal cannula is provided at high flow rates, optionally up to about 120 L/min, optionally about 10-70 L/min.

In some configurations, the gases flow provided by the non-sealing nasal cannula is humidified.

In accordance with certain features, aspects and advantages of at least one of the embodiments disclosed herein, a mask configured to substantially surround an opening of a patient's airway is provided, the mask comprising: a frame configured to position on an upper portion of the patient's face above the patient's nares; and a fabric extending from a lower portion of the frame and configured to accommodate an opening of the patient's airway in use, the fabric configured to enable a flow of gases from a patient facing side of the mask to a non-patient facing side of the mask in use.

In some configurations, the fabric is configured to accommodate the patient's nose and mouth in use.

In some configurations, the fabric is configured to drape over the patient's face.

In some configurations, the fabric comprises a material that is permeable to water molecules and gases flow.

In some configurations, the fabric comprises a material that is substantially impermeable to bulk flow of liquid water.

In some configurations, the fabric is hydrophilic, optionally wherein the fabric comprises a hydrophilic material or comprises a material that has been treated to be hydrophilic.

In some configurations, the fabric comprises an absorbent material.

In some configurations, the fabric is removably connected to the frame at the lower portion, optionally via a hook-and-loop fastener system.

In some configurations, the frame is configured to extend across the patient's orbital region in use.

In some configurations, the frame comprises a transparent material.

In some configurations, the frame is configured to extend to the sides of the patient's head.

In some configurations, the frame comprises a retention mechanism to retain the mask on the patient's head.

In some configurations, the frame comprises frame ends configured to loop behind the patient's ears to secure the mask on the patient's face.

In some configurations, the frame is configured to extend to the rear of the patient's head.

In some configurations, the frame comprises a pad that is configured to seal about or against the patient's face.

In some configurations, the lower portion of the frame comprises the pad.

In accordance with certain features, aspects and advantages of at least one of the embodiments disclosed herein, an interface system is provided, the interface system comprising: a non-sealing patient interface configured to deliver a gases flow to a patient's airway; and the mask as outlined above.

In some configurations, the non-sealing patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s).

In some configurations, the non-sealing nasal cannula comprises a cannula headgear configured to retain said non-sealing nasal cannula on the patient's face in use.

In some configurations, the fabric of the mask is configured to accommodate a portion of the non-sealing patient interface.

In some configurations, the fabric of the mask is configured to drape over a portion of the non-sealing patient interface.

In some configurations, the mask comprises a mask headgear, wherein the cannula headgear and mask headgear are separate such that the mask is selectively securable to or removable from the patient independently of the non-sealing patient interface.

In some configurations, the mask does not comprise a gases supply or gases removal conduit and/or does not comprise a connection port for connecting to a gas source or gas removal device and/or is not adapted to actively deliver a flow of gases to the patient and/or is not adapted to actively remove a flow of gases from the patient.

In some configurations, the gases flow to the patient is only provided by the nasal cannula.

In some configurations, the non-sealing patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s), and wherein the gases flow provided by the nasal cannula is provided at high flow rates, optionally up to about 120 L/min, or about 10-70 L/min.

In some configurations, the gases flow provided by the non-sealing nasal cannula is humidified.

In accordance with certain features, aspects and advantages of at least one of the embodiments disclosed herein, a mask configured to substantially surround an opening of a patient's airway is provided, the mask comprising a mask body having a preformed shape that is configured to deflect fluids from the opening of a patient's airway in an intended direction; and either: the mask further comprises an interfacing feature configured to, in use, interface with a patient interface provided on the patient; or the mask is integrally formed with a patient interface.

In some configurations, the patient interface comprises one or more venting apertures, and the mask is configured to filter and/or deflect fluids that exit the patient interface via the venting aperture(s).

In some configurations, the patient interface comprises a non-sealing patient interface.

In some configurations, the patient interface comprises a nasal cannula.

In some configurations, the patient interface comprises a non-sealing nasal cannula comprising one or more prongs that extend(s) into the patient's nasal passage(s) in use, side arms that extend laterally along the patient's face in use, and a patient conduit adapted to connect to a gases source for delivering a gases flow to the patient via the prong(s).

In some configurations, the interfacing feature comprises a clip that is configured to couple to a manifold and/or patient conduit of the nasal cannula.

In some configurations, the clip is configured so that the mask is rotatable relative to the nasal cannula about an axis through a length of a body of the nasal cannula.

In some configurations, the mask body comprises a domed shape in at least one plane.

In some configurations, the mask body comprises a generally triangular shape when viewed from a front of the mask.

In some configurations, the mask body comprises a concave shape when viewed from a rear of the mask.

In some configurations, edges of the mask body are smooth and/or wherein corners of the mask body are rounded.

In some configurations, the mask body comprises a filter and/or absorbent material.

In some configurations, the mask body is rigid.

In some configurations, the mask body has a thickness of about 1 mm or less.

In some configurations, the mask body is inflatable.

In some configurations, the mask has a light weight.

In some configurations, the mask is adapted to direct fluids towards the patient's chest.

In some configurations, the mask is integrally formed with the patient interface.

In some configurations, the mask body comprises a plurality of body portions that function together to deflect the fluid.

In some configurations, the body portions are angled differently from each other.

Features from one or more embodiments or configurations may be combined with features of one or more other embodiments or configurations.

As used herein the term ‘(5)’ following a noun means the plural and/or singular form of that noun.

As used herein the term ‘and/or’ means ‘and’ or ‘or’, or where the context allows both.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

This disclosure may also be said broadly to consist in the parts, elements and features referred to or indicated in the specification of the application, individually or collectively, and any or all combinations of any two or more said parts, elements or features.

Where specific integers are mentioned herein which have known equivalents in the art to which this disclosure relates, such known equivalents are deemed to be incorporated herein as if individually set forth.

The disclosure consists in the foregoing and also envisages constructions of which the following gives examples only.

### BRIEF DESCRIPTION OF THE DRAWINGS

Specific embodiments and modifications thereof will become apparent to those skilled in the art from the detailed description herein having reference to the figures that follow, of which:

FIG. 1 is a schematic view of an overall respiratory assistance system.

FIG. 2 is a perspective view of an exemplary first configuration patient interface that can be used with the masks described herein.

FIG. 3 is a perspective view of an exemplary second configuration patient interface that can be used with the masks described herein.

FIG. 4 shows a first configuration mask, where FIG. 4(a) is a front perspective view and FIG. 4(b) is a rear perspective view.

FIG. 5 is a perspective view of the first configuration mask and exemplary patient interface on the face of a patient.

FIG. 6 is a perspective view of a second configuration mask.

FIG. 7 is a perspective view of a third configuration mask and exemplary patient interface on the face of a patient.

FIG. 8 is a perspective view of a fourth configuration mask and exemplary patient interface on the face of a patient.

FIG. 9 is an exploded perspective view of the fourth configuration mask and exemplary patient interface.

FIG. 10 is a perspective view of a fifth configuration mask.

FIG. 11 is a perspective view of a sixth configuration mask.

FIG. 12 is a perspective view of a seventh configuration mask and exemplary patient interface on the face of a patient.

FIG. 13 is an exploded perspective view of the seventh configuration mask and exemplary patient interface.

FIG. 14 is a perspective view of an eighth configuration mask and exemplary patient interface on the face of a patient.

FIG. 15 is a perspective view of a ninth configuration mask and exemplary patient interface on the face of a patient.

FIG. 16 is a perspective view of a tenth configuration mask and exemplary patient interface on the face of a patient.

FIG. 17 is a perspective view of an eleventh configuration mask and exemplary patient interface on the face of a patient.

FIG. 18 is a perspective view of a twelfth configuration mask and exemplary patient interface on the face of a patient.

FIG. 19 is a rear perspective view of a thirteenth configuration mask.

FIG. 20 is a front perspective view of the thirteenth configuration mask and an exemplary patient interface during application to the face of a patient.

FIG. 21 is a front perspective view of the thirteenth configuration mask and exemplary patient interface once applied to the face of a patient.

FIG. 22 is a perspective view of a fourteenth configuration mask and exemplary patient interface on the face of a patient.

FIG. 23 is a perspective view of a fifteenth configuration mask and exemplary patient interface on the face of a patient.

FIG. 24 is a perspective view of a sixteenth configuration mask and exemplary patient interface on the face of a patient.

FIG. 25 is a perspective view of a seventeenth configuration mask and exemplary patient interface on the face of a patient.

FIG. 26 is an exploded perspective view of the seventeenth configuration mask and exemplary patient interface.

FIG. 27 is a perspective view of an eighteenth configuration mask and exemplary patient interface on the face of a patient.

FIGS. 28(a) and 28(b) are partial perspective views of a first alternative configuration interfacing feature for the mask of FIG. 27.

FIGS. 29(a) and 29(b) are partial perspective views of a second alternative configuration interfacing feature for the mask of FIG. 27.

FIGS. 30(a) and 30(b) are partial perspective views of a third alternative configuration interfacing feature for the mask of FIG. 27.

FIGS. 31(a) and 31(b) are partial perspective views of a fourth alternative configuration interfacing feature for the mask of FIG. 27.

FIG. 32 is a perspective view of a nineteenth configuration mask and exemplary patient interface on the face of a patient.

FIG. 33 is a perspective view of a twentieth configuration mask and exemplary patient interface on the face of a patient.

FIG. 34 is a perspective view of a twenty-first configuration mask and exemplary patient interface on the face of a patient.

FIG. 35 is a perspective view of a twenty-second configuration mask and exemplary patient interface on the face of a patient.

FIG. 36 is a perspective view of a twenty-third configuration mask and a patient wearing an exemplary patient interface.

FIG. 37 is a perspective view of the twenty-third configuration mask and exemplary patient interface on the face of a patient.

FIG. 38 is a perspective view of a twenty-fourth configuration mask (showing the back of the mask) and a patient wearing an exemplary patient interface.

FIG. 39 is a perspective view of the twenty-fourth configuration mask and exemplary patient interface on the face of a patient.

FIG. 40 is a side view of a twenty-fifth configuration mask and exemplary patient interface on the face of a patient.

FIG. 41 is a side sectional view of the twenty-fifth configuration mask and exemplary patient interface on the face of a patient.

FIG. 42 is a side view of a twenty-sixth configuration mask and exemplary patient interface on the face of a patient.

FIG. 43 is a side sectional view of the twenty-sixth configuration mask and exemplary patient interface on the face of a patient.

FIG. 44 is a perspective view of a twenty-seventh configuration mask and exemplary patient interface on the face of a patient.

FIG. 45A is a perspective view of an alternative twenty-seventh configuration mask and exemplary patient interface on the face of a patient.

FIG. 45B is a perspective view of another alternative twenty-seventh configuration mask and exemplary patient interface on the face of a patient.

FIG. 45C is a perspective view of yet another alternative twenty-seventh configuration mask and exemplary patient interface on the face of a patient.

FIG. 46 is a perspective view of a twenty-eighth configuration mask and exemplary patient interface.

FIG. 47A is a side view of an exemplary patient interface on the face of a patent, showing expired fluid flow without a mask.

FIG. 47B is a side view similar to FIG. 47A, but showing deflection of the expired fluid flow using the twenty-eighth configuration mask.

### DETAILED DESCRIPTION

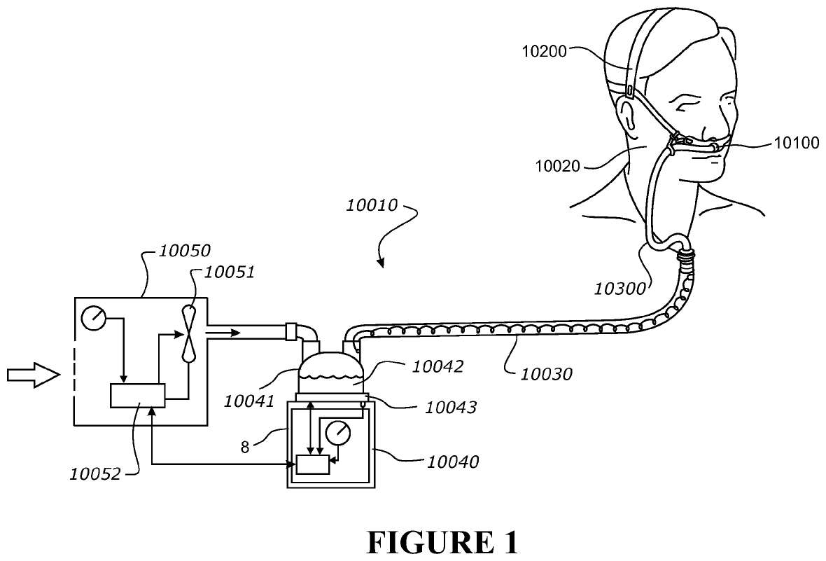

Referring to FIG. 1, a ventilation and humidification system (a respiratory assistance system 10010) that may be used with the masks of the present disclosure is shown. In such a system 10010, a patient 10020 is supplied with a flow of gases through a patient interface 10100. The flow of gases may be humidified. The patient interface 10100 is retained in an operational position upon the patient's face using associated headgear 10200. The headgear 10200 provides a first retention mechanism configured to retain the patient interface 10100 on the patient's face in use.

The patient interface 10100 is connected to a humidified gases transportation pathway or inspiratory conduit 10030. The inspiratory conduit 10030 is connected at one end (either directly or indirectly) to the patient interface 10100 and at an opposing end to the outlet of a humidifier 10040. In the preferred embodiment the inspiratory conduit is connected to the patient interface via an extension tube/conduit 10300. The extension tube/conduit 10300 and/or the inspiratory conduit 10030 form a patient conduit adapted to connect to a gases source for delivering a gases flow to the patient.

The extension tube/conduit 10300 can be considered to be part of the patient interface. Therefore, as used herein, “patient interface” can be considered to be not only the portion of the interface that contacts and/or interfaces with the patient, but additionally the conduit that delivers the flow of gases to the interface (such as extension tube/conduit 10300 for example).

The humidifier 10040 receives and humidifies gas supplied from a flow generator or gases source 10050, which may include a blower 10051. Alternatively, other gases sources could be used, such as a wall source with a flow meter. The humidifier 10040 and gases source 10050 may be integrated or may be in separate modules. The humidifier 10040 may comprise a humidification chamber 10041 filled with water or other liquid 10042 and a heating means 10043 for heating the water to humidify the gas path through the humidifier. A controller 10052 may be provided to control and possibly vary one or more properties of the supplied gas, including but not limited to the pressure profile of the gas, the flow rate profiles of the gas at the patient interface, the temperature of the gas and/or the humidity of the gas. It will be appreciated that the control capabilities are dependent on the purpose and application of the respiratory assistance system 10010. For example, in the preferred application of in-hospital respiratory care, the flow rate of supplied gas is monitored and controlled according to the patient's requirements but the pressure of the supplied gas is not necessarily monitored and controlled.

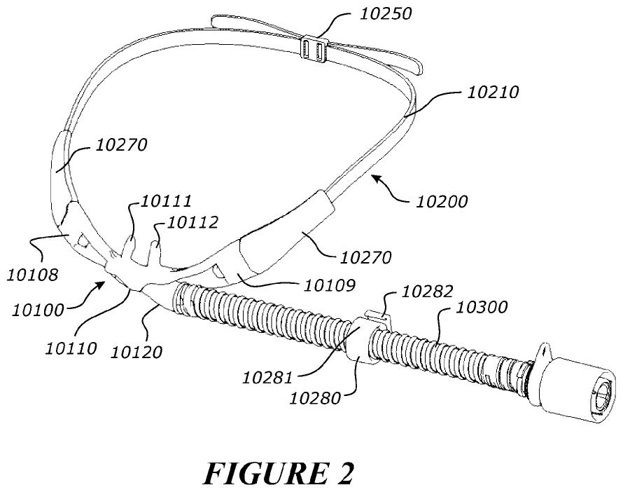

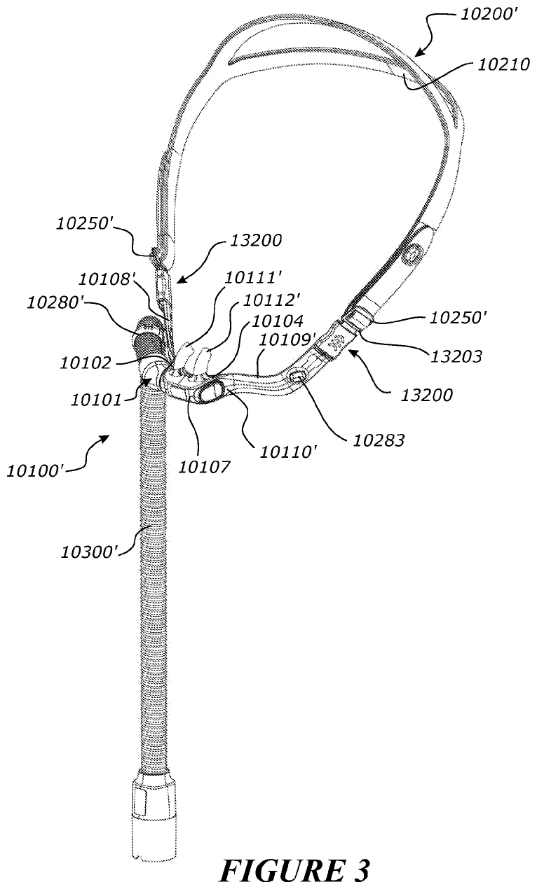

The respiratory system 10010 may be a high flow therapy apparatus or system. High flow therapy as discussed herein is intended to be given its typical ordinary meaning as understood by a person of skill in the art, which generally refers to a respiratory assistance system delivering a targeted flow of humidified respiratory gases via an intentionally unsealed patient interface with flow rates generally intended to meet or exceed inspiratory flow of a patient. Typical patient interfaces include, but are not limited to, a nasal or oral patient interface. Typical flow rates for adults often range from, but are not limited to, about fifteen litres per minute (LPM) to about seventy litres per minute or greater. Typical flow rates for pediatric patients (such as neonates, infants and children) often range from, but are not limited to, about one litre per minute per kilogram of patient weight to about three litres per minute per kilogram of patient weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), among other common names. The flow rates used to achieve ‘high flow’ may be any of the flow rates listed below. Medicament(s) or drug(s) can be introduced to the respiratory system 10010 and hence to a patient 10020 via one or more of: at or adjacent an exit port of the humidification chamber 10041; between the inspiratory conduit 10030 or extension tube/conduit 10300 and the patient interface 10100, 10100′ (FIGS. 2 and 3); into the patient interface 10100, 10100′, including at or adjacent nasal prongs 10111, 10112, 10111′, 10112′, the manifold assembly 10101 and/or the gases flow manifold part 10120.

For example, in some configurations, for an adult patient ‘high flow therapy’ may refer to the delivery of gases to a patient at a flow rate of greater than or equal to about 10 litres per minute (10 LPM or 10 L/min), such as between about 10 LPM and about 120 LPM, or between about 10 LPM and about 100 LPM, or between about 15 LPM and about 95 LPM, or between about 20 LPM and about 90 LPM, or between 25 LPM and 75 LPM, or between about 25 LPM and about 85 LPM, or between about 30 LPM and about 80 LPM, or between about 35 LPM and about LPM, or between about 40 LPM and about 70 LPM, or between about 45 LPM and about 65 LPM, or between about 50 LPM and about 60 LPM. In some configurations, for a neonatal, infant, or child patient ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may deliver gases to the patient at a flow rate of between about 1 LPM and about 100 LPM, or at a flow rate in any of the sub-ranges outlined above. The flow therapy apparatus 10 can deliver any concentration of oxygen (e.g., FdO2), up to 100%, at any flowrate between about 1 LPM and about 100 LPM. In some configurations, any of the flowrates can be in combination with oxygen concentrations (FdO2s) of about 20%-30%, 21%-30%, 21%-40%, 30%-40%, 40%-50%, 50%-60%, 60%-70%, 70%-80%, 80%-90%, and 90%400%. In some combinations, the flow rate can be between about 25 LPM and 75 LPM in combination with an oxygen concentration (FdO2) of about 20%-30%, 21%-30%, 21%-40%, 30%-40%, 40%-50%, 50%-60%, 60%-70%, 70%-80%, 80%-90%, and 90%400%. In some configurations, the flow therapy apparatus 10 may include safety thresholds when operating in manual mode that prevent a user from delivering to much oxygen to the patient.

It will be appreciated that LPM and ‘L/min’ can be used interchangeably to mean ‘litres per minute’.

High flow therapy may be administered to the nares of a user and/or orally. High flow therapy may deliver gases to a user at a flow rate at or exceeding the intended user's peak inspiratory flow requirements. The high flow therapy may generate a flushing effect in the nasopharynx such that the anatomical dead space of the upper airways is flushed by the high incoming gases flow. This can create a reservoir of fresh gas available for each and every breath, while minimizing re-breathing of nitrogen and carbon dioxide. Meeting inspiratory demand and flushing the airways is additionally important when trying to control the patient's FdO2. High flow therapy can be delivered with a non-sealing patient interface such as, for example, a nasal cannula. The nasal cannula may be configured to deliver breathing gases to the nares of a user at a flow rate exceeding the intended user's peak inspiratory flow requirements.

Nasal high flow provides dynamic pressure to a patient in synchrony to the breathing of the patient. For example, nasal high flow being provided to a patient can increase pressure during the expiratory phase of a patient. This can reduce the respiratory rate of the patient and reduce respiratory effort of the patient. Reduced respiratory effort and respiratory rate are helpful to a patient with respiratory conditions e.g. COPD.

The term ‘non-sealing patient interface’ as used herein can refer to an interface providing a pneumatic link between an airway of a patient and a gases flow source that does not completely occlude the airway of the patient. Non-sealed pneumatic link can comprise an occlusion of less than about 95% of the airway of the patient. The non-sealed pneumatic link can comprise an occlusion of less than about 90% of the airway of the patient. The non-sealed pneumatic link can comprise an occlusion of between about 40% and about 80% of the airway of the patient. The airway can include one or more of a nare or mouth of the patient. For a nasal cannula the airway is through one or both of the nares.

Referring to FIG. 2, an exemplary form of the patient interface 10100 is shown. The patient interface 10100 is configured to deliver breathing gases from a gases supply and humidification source (e.g. as shown in FIG. 1) to the patient, and a patient interface headgear 10200 is configured to support and retain the patient interface against the patient's face in use.

The patient interface may be a non-sealing patient interface. The patient interface may be a non-sealing oral and/or nasal patient interface.

The non-sealing patient interface may be unsealed from a patient's airway opening in use, or may be partly-sealed with a patient's airway opening in use.

In one exemplary configuration, the non-sealing patient interface may be a non-sealing patient interface that is configured to substantially seal with a single one of the patient's nares, with the other one of the patient's nares being left unsealed and free. An exemplary non-sealing patient interface of that type is disclosed in U.S. provisional patent application Nos. 62/777,721, 62/826,529, 62/910,702, and PCT application number PCT/IB2019/060589 (published as PCT publication number WO 2020/121177) to Fisher & Paykel Healthcare Limited. The contents of those specifications are incorporated herein in their entireties by way of reference.

Two other exemplary configurations are described below.

The patient interface 10100 of the exemplary configuration is in the form of a non-sealing nasal cannula 10100 that is adapted to couple an inspiratory conduit 10300 and that comprises at least one, but preferably two, nasal prongs 10111 and 10112 configured to fit within the nares of a patient to deliver a flow of gases to the patient. The headgear 10200 is in the form of a head strap 10200 that is preferably adjustable in length to customise the size of the strap to the patient.

The nasal cannula 10100 provides a patient with a patient interface suitable for the delivery of high flow, high humidity gas flow to the patient's nasal cavity/nares. In some configurations, the cannula is adapted to deliver a high flow of gases over a wide flow range (e.g. may be 10-70 L/min, may be 100 L/min, or may be up to about 120 L/min.

The nasal cannula 10100 comprises a face mount part 10110 including at least one, but preferably a pair of tubular nasal prongs 10111 and 10112, integrally moulded with or removably attached to the face mount part 10110, and a gases flow manifold part 10120 that is removably attached or integrally moulded to the conduit 10300. The gases flow manifold part 10120 is insertable into the face mount part from either one of two opposing horizontal directions, i.e. from either left side or the right side. In this manner, the position or location of the gases flow manifold part 10120 is reversible with respect to the face mount part 10110. In other words, a user may choose to have the manifold part 10120 and the conduit 10300 extending therefrom extend from either the left side or the right side of the cannula 10100 depending on what is most convenient, for example depending on which side of the user the gas source or ventilator is located.

The face mount part 10100 is formed from a soft, flexible and material such as silicone or another cannula material known in the art. The nasal prongs 10111 and 10112 may be supple and may be formed from a sufficiently thin layer of silicone to achieve this property.

The gases flow manifold part 10120 is formed from a relatively harder material such as Polycarbonate, a High-Density Polyethylene (HDPE) or any other suitable plastics material known in the art. The face mount part 10110 provides a soft interfacing component to the patient for comfortably delivering the flow of gases through the nasal prongs 10111 and 10112, while the gases flow manifold part 10120 fluidly couples the conduit 10300 to the nasal prongs 10111 and 10112 of the face mount part 10110.

The nasal prongs 111 and 112 may be curved to extend into the patient's nares in use and to provide a smooth flow path for gases to flow through. The inner surfaces of the prongs 10111 and 10112 may be contoured to reduce noise. The bases of the prongs 10111 and 10112 may include curves surfaces to provide for smoother gases flow. This may reduce the noise level during operation.

In some configurations, pads may be mounted around the base of the prongs to reduce noise. The pad may be a foam material or a mouldable material that generally conforms to the patient's nose anatomy. Soft cushions or pillows may alternatively be provided.

The nasal prongs 10111 and 10112 are substantially hollow and substantially tubular in shape.

The nasal prongs 10111 and 10112 may be consistent in diameter along their lengths but are may alternatively be shaped to fit the contours of the nares. Each prong 10111, 10112 has an elongate opening (not shown) at the distal end opposing a base portion 10118 of the face mount part 10110 to encourage a high flow of gases into the cavity. In alternative embodiments, the nasal prongs 10111 and 10112 may have a tapered profile of a wider end at the base portion 10118 and a narrower end at the openings.

The face mount portion 10110 and in particular the nasal prongs 10111 and 10112 are designed not to seal about the patient's nares to avoid excessive and potentially harmful build-up of pressure during high flow therapy. The nasal prongs 10111 and 10112 are therefore sized to maintain a sufficient gap between the outer surface of the prongs 10111 and 10112 and the patient's skin to avoid sealing the gas path between the cannula 10100 and patient. The cannula 10100 is therefore a non-sealing nasal cannula.

The face mount part 10110 is shaped to generally follow the contours of a patient's face around the upper lip area. The face mount part 10100 is moulded or pre-formed to be able to conform to and/or is pliable to adapt, accommodate and/or correspond with the contours of the user's face, in the region of the face where the cannula is to be located.

The face mount part 10110 of the nasal cannula has side arms 10108, 10109 that extend laterally along the patient's face in use.

A retention clip 10280 may comprise a tubular body 10281 for receiving and accommodating a portion of the conduit 10300. A hook 10282 projects from the body 10281. Via this hook 10282, the conduit 10300 can be coupled or tethered to the head strap 10210 or headgear 10200 in use.

The headgear 10200 may have different configurations. For example, the headgear 10200 may have a strap 10210 to extend around the back of a patient's head, and may have a buckle 10250 to enable adjustment of the length of the strap.

Sleeves 10270 may couple ends of the headgear 10200 to the side arms 10108, 10109.

The nasal cannula 10110, headgear 10200, and/or respiratory assistance system 10010 may be of the type described in PCT application number PCT/NZ2014/000082 (published as PCT publication number WO 2014/182179) and United States patent application publication 2019/0344036 A1 to Fisher & Paykel Healthcare Limited. The contents of those specifications are incorporated herein in their entireties by way of reference.

The nasal cannula 10110 may, for example, be the Optiflow™+ non-sealing nasal cannula provided by Fisher & Paykel Healthcare Limited of Auckland, New Zealand.

FIG. 3 shows an alternative configuration patient interface in the form of a non-sealing nasal cannula 10100′ that may be used with the system of FIG. 1 and/or with the masks disclosed herein. Unless described as otherwise below, the features and functionality may be considered the same as those of the nasal cannula of FIG. 2, and like reference numbers indicate like parts with the addition of a prime (′).

The side arms 10108, 10109′ comprise headgear retaining mechanisms adapted to hold headgear 10200′. In the illustrated embodiment of FIG. 3 the retaining mechanism are buckles or connectors 13200 comprising a first connector part in the form of a clip (not shown) and a second connector part in the form of a carrier 13203.

The patient interface 10100′ comprises a frame portion 10102. The frame portion 10102 comprises a contact region 10104 that contacts a patient in use. At least a part of the contact region 10104 sits under a nose or under nares of a patient in use (for example, on the lip superior). The frame portion 10102 also comprises a non-contact region 10107 that faces away from the patient in use. In the illustrated configuration, the non-contact region 10107 is formed from a relatively hard or rigid material (for example polycarbonate and/or polypropylene) that provides support to the frame portion 10102.

The configuration of FIG. 3 is provided with a manifold assembly 10101 that may be arranged to position the conduit/tube 10300′ to either the left or right side of the patient interface. The configuration of FIG. 3 comprises a manifold 10101 that may be pushed into or received in a gases chamber of the frame portion 10102. In some embodiments, the manifold 10101 may be received in the gases chamber in two orientations. If a user desires the conduit to extend from the patient interface on the right hand side then the manifold assembly 10101 may be inserted into the chamber via a first gas inlet. Alternatively, if the user desires the conduit to extend from the interface on the left hand side then the manifold assembly 10101 may be inserted into the chamber via a second gas inlet.

The patient interface 10100′ comprises a side arm mount 10283 on the frame 10102 of the patient interface, to engage with the tube clip 10280′. The frame 10102 may comprise a side arm mount 10283 on each of the first and second arms 10108′, 10109′ to interface with component 10280′ to hold the tube either to the left or right hand side of the patient interface. The side arm mount(s) could be any suitable configuration, such as an aperture or recess for example.

The headgear 10200′ of this patient interface has two buckles 10250′, one on either side of the nasal cannula 10100′, to enable adjustment of the length of the headgear strap 10210′.

The nasal cannula 10110′, headgear 10200′, and/or respiratory system 10010 may be of the type described in PCT application number PCT/IB2015/054585 (published as PCT publication number WO 2015/193833) and United States patent application publication number 2021/0077764 A1 to Fisher & Paykel Healthcare Limited. The contents of those specifications are incorporated herein in their entireties by way of reference.

The nasal cannula 10110′ may, for example, be the Optiflow™ 3S non-sealing nasal cannula provided by Fisher & Paykel Healthcare Limited of Auckland, New Zealand.

During application of nasal high-flow (NHF) therapy, the air exhaled from a patient may travel a significant distance depending on the therapy flow rate. When a patient coughs, this distance is further increased. This exhaled air may contain contaminants (includes pathogens) and aerosols that are undesirable for other persons who may be in proximity to the patient.

The masks disclosed herein are configured to greatly reduce the dispersal of exhaled contaminants and aerosols.

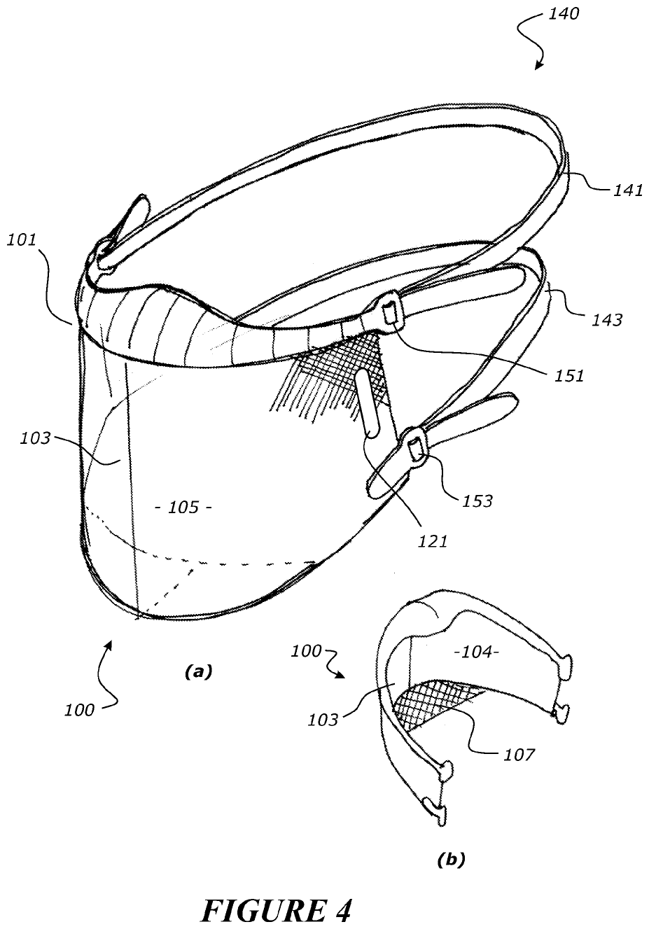

FIGS. 4 to 35 show masks that can be used with patient interfaces such as the non-sealing nasal cannulas 10110, 10110′ or any other suitable non-sealing patient interface to provide interface systems. The patient interface may be a non-sealing oral and/or nasal patient interface. The masks may be provided separately from the patient interfaces, or may be provided in interface systems with the patient interfaces.

Many of the disclosed masks can interface with an underlying patient interface and many of the disclosed masks comprise a filter to remove pathogens, contaminants, and/or aerosols from a fluid that travels from within the mask to ambient. The fluid may comprise liquid, gas, or a combination thereof.

The masks may be full-face masks, nasal masks, oral masks, or any covering that can be provided on another patient interface—e.g. a covering surrounding a portion of the patient interface such as the nasal prong(s).

The masks are configured to substantially surround an opening of a patient's airway, such as the patient's mouth and/or nose.

The masks are configured to be worn concurrently with a non-sealing respiratory patient interface. At least some of the masks can filter fluid emanating from a patient's nose and/or mouth or that has condensed on the surface of the non-sealing patient interface. This may involve filtering pathogens (including viral particles) or nebulised drugs. During high flow therapy, condensate can readily form from exhaled air and/or from the humidified gases provided to the patient, proximal to the patient interface. Covering the mouth is beneficial for situations in which a patient coughs or sneezes or otherwise exhales aerosols out of the mouth.

The masks (and generally the mask bodies and/or filters) are configured to enable a flow of gases from a patient facing side of the masks to a non-patient facing side of the masks. The masks are configured so as to not substantially impede the flow rate of exhaled gases, to avoid any increasing of pressure inside the masks which could potentially cause lung damage and CO₂ build-up.

The masks are described and shown in different configurations. A skilled person will, however, appreciate that features from one or more configurations can be used with features from one or more other configurations, or different combinations of features can be combined in other configurations.

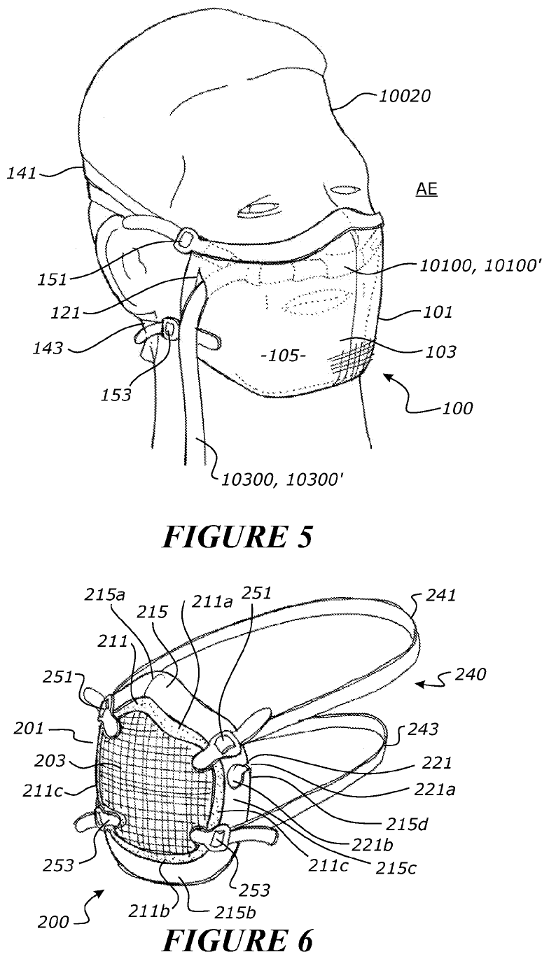

A first configuration mask 100 is shown in FIGS. 4 and 5. The mask 100 is configured to substantially surround an opening of a patient's airway.

The mask has a mask body 101 comprising a filter 103 configured to filter a fluid from a patient facing side 104 of the mask to a non-patient facing side 105 of the mask. The non-patient facing side 105 of the mask faces an ambient environment AE.

The filter 103 of the mask body 101 is configured to enable a flow of gases from the patient facing side 104 of the mask to the non-patient facing side 105 of the mask.

The mask 100 further comprises an interfacing feature 121 configured to, in use, interface with a patient interface provided on the patient 10020.

In the figures in this specification the filter 103, 503, 703, 803, 903, 1003, 1103, 1203, 1403, 1503, 1603, 1903, 2003, 2103, 2203, 2303, 2403 on the mask body 101, 501, 701, 801, 901, 1001, 1101, 1201, 1401, 1501, 1601, 1901, 2001, 2101, 2201, 2301, 2401 is shown schematically as a hatched region on only a portion of the mask body to simplify the drawings. However, it will be appreciated for any of the configurations herein, the mask body may be substantially formed of the filter so the filter covers substantially the entire mask body, or the filter may cover a portion of the mask body 101, 501, 701, 801, 901, 1001, 1101, 1201, 1401, 1501, 1601, 1901, 2001, 2101, 2201, 2301, 2401.

The mask body 101 and/or filter 103 may comprise a pre-formed shape. For example, to generally follow the contours of a patient's face. In some configurations, the mask body and/or filter is rigid or semi-rigid. Alternatively, the filter 103 may be provided on an underlying material that imparts a pre-formed shape to the filter.

Suitable materials for the filter 103 comprise one or more of polyester, polyurethane, nylon, or surface-modified polypropylene, natural fibres, cotton, wool, hemp, and/or bamboo for example.

Suitable materials for the underlying material may comprise one or more of polycarbonate, polypropylene, or acrylonitrile butadiene styrene (ABS).

The mask body 101 is configured to substantially cover a front portion of the patient's face. The mask body 101 is optionally configured to substantially cover side portions of the patient's face.

The mask body 101 also has a chin portion 107 that extends across the bottom of the mask body 101 and under a patient's chin in use. The chin portion 107 may be formed of the filter 103 or may alternatively be formed of a different material, such as an elastic material for example. The chin portion may be a sewn-in elastic fabric or may be formed from the same material as the rest of the mask body.

In some configurations, the chin portion 107 allows tightening of the mask. The chin portion 107 may keep the patient's mouth closed during use of the mask.

The filter 103 is configured to limit travel of a liquid from the patient-facing side 104 of the mask to the non-patient facing side 105 of the mask. For example, if fluid expelled from a patient's airway contains gas and a liquid, the filter 103 will limit travel of the liquid from the patient-facing side 104 of the mask to the non-patient facing side. The liquid may, for example, form from condensation or may otherwise emerge from a patient's oronasal region.

The filter 103 may comprise a material that is permeable to water molecules and gases flow.

The material may be substantially impermeable to bulk flow of liquid water.

The mask 100 may comprise an absorbent material to absorb the liquid from the fluid expelled from the patient's airway. The absorbent material can capture the aerosols from the patient, e.g. when a patient coughs and sneezes, and will advantageously prevent droplets from accumulating within the mask.

The filter 103 may comprise the absorbent material. Alternatively, the absorbent material may be in addition to the filter 103.

The absorbent material may be hydrophilic. For example, the absorbent material may comprise a hydrophilic material or alternatively may comprise a material that has been treated to be hydrophilic.

In one configuration, the absorbent material is configured to wick the liquid away from the patient. Wicking involves capillary action to move the liquid. In an alternative configuration, the absorbent material is configured to permit evaporation of the liquid away from the patient, optionally to the ambient environment.

Small water molecules may pass through the absorbent material for evaporation, but larger adverse particles such as viral particles or other pathogens or contaminants may be captured by the absorbent material and unable to pass through the thickness of the absorbent material the way water molecules can.

The absorbent material may comprise one or more of titanium dioxide, acetic acid, silver nanoparticles, copper, or zinc, which are useful in the inactivation of pathogens.

The absorbent material may comprise any suitable material such as, for example, one or more of polyester, polyurethane, nylon, polyethylene or a composite thereof.

The filter 103 may comprise a fabric, such as a textile fabric and/or a polymer matrix fabric for example. Suitable materials comprise one or more of polyester, polyurethane, nylon, or surface-modified polypropylene, natural fibres, cotton, wool, hemp, and/or bamboo for example.

In some configurations, the mask body 101 and/or filter 103 may comprise an elastic material, to enable the mask body and/or filter to flex to differently sized or shaped patient's faces.

In some configurations, the mask body 101 and/or filter 103 is/are configured to minimize impediment on flow rates of a gases flow through the mask body and/or filter. This enables the mask 100 to be used in high flow therapy without substantially increasing pressure in the mask to avoid any possibility of lung damage from pressure build-up and CO₂ build-up.

In some configurations, the mask body 101 and/or filter 103 is configured to allow a flow rate of a gases flow through it of up to about 450 L/min to accommodate patient coughing or sneezing without pressure build-up on the patient-facing side of the mask. In some configurations, the mask body 101 and/or filter 103 is configured to allow a flow rate of a gases flow through the mask body and/or filter of about 10-450 L/min to accommodate patient coughing.

The mask body 101 and/or filter 103 can absorb aerosols and/or can trap pathogens and/or contaminants with minimal or no pressure therapy contribution.

The filter 103 may be porous to allow air to allow air to pass through it, to avoid undesirable increases of pressure inside the mask 100. Open-to-closed ratios of the filter 103 material may be selected to achieve a desired level of porosity. Additionally, or alternatively, the mask body 101 may comprise arrangements of closed portion(s) and filter portion(s) to provide an overall desired level of porosity of the mask.

Porosity of the filter 103 will enable exhaled flow to pass and minimise carbon dioxide build-up whilst facilitating dead space clearance.

The mask 100 is appropriately sized to cover at least part of the patient interface 10100, 10100′ in use. As outlined above, the patient interface 10100, 10100′ may be a non-sealing patient interface.

The patient interface 10100, 10100′ comprises a non-sealing nasal cannula comprising one or more prongs 10111, 10112, 10111′, 10112′ that extend(s) into the patient's nasal passage(s) in use, side arms 10108, 10109, 10108′, 10109′ that extend laterally along the patient's face in use, and a patient conduit 10300, 10300′ adapted to connect to a gases source for delivering a gases flow to the patient via the prong(s).

In the configuration shown, the mask 100 is adapted to cover the nose and mouth of the patient.

The mask body 101 may have a surface area or volume that is sufficient to deal with saturation arising from condensate formation.

The mask 100 can act acts as reservoir to hold liquid, and the mask can be disposed of after capturing liquid.

The mask can be configured in such a way that it allows sufficient evaporation from the non-patient facing side 104 of the mask to maintain patient comfort and minimise condensation build-up.

The mask body 101 may be composed of multiple layers, with different layers exhibiting different characteristics.

The mask 100 does not comprise a gases supply or gases removal conduit and/or does not comprise a connection port for connecting to a gas source or gas removal device and/or is not adapted to actively deliver a flow of gases to the patient and/or is not adapted to actively remove a flow of gases from the patient. That is, gases flow is only delivered to the patient via the patient interface 10100, 10100′ rather than via the mask 100. The purpose of the mask 100 is to filter fluids from the patient rather than delivering gases to the patient.

The mask comprises an interfacing feature 121 configured to interface with the cannula when donned on the patient. The interfacing feature may interact with the cannula body, cannula tube or conduit, or any ancillary component of the cannula.

The interfacing feature 121 comprises an aperture provided in the mask body 101 to allow a portion of the nasal cannula 10100, 10100′ to extend through the mask body. In the configuration shown, the interfacing feature comprises an aperture 121 that allows the patient conduit 10300, 10300′ of the nasal cannula to extend through the aperture. Additionally, or alternatively, the aperture 121 could allow one of the side arms 10108, 10108′, 10109, 10109′ of the nasal cannula 10100, 10100′ to extend through the aperture.

The aperture 121 may be provided in a side of the mask body 101. As shown in FIGS. 4 and 5, in some configurations two of said apertures 121 may be provided. The two apertures 121 may be provided in opposing sides of the mask body 101.

The mask 100 comprises a second retention mechanism configured to retain the mask 100 on the patient's face in use. The second retention mechanism of the mask may be separate from the first retention mechanism of the patient interface, such that the mask 100 is separately securable to or removable from the patient independent of the non-sealing patient interface. This enables the removal of the mask 100 from the patient while leaving the nasal cannula 10100, 10100′ on the patient, without stopping or affecting therapy.

In the configuration shown, the second retention mechanism of the mask 100 comprises a headgear 140 and/or headgear connection features 151, 153 to secure the mask to the patient independently of the nasal cannula 10100, 10100′. The headgear 140 may be integral with the mask 100 or may be releasably connectable to the headgear connection features 151, 153.

In the configuration shown, the mask comprises a pair of upper headgear connection features 151, one on either side of the mask body 101 at or adjacent a rear edge of the mask body. The mask further comprises a pair of lower headgear connection features 153, one on either side of the mask body 101 at or adjacent a rear edge of the mask body.

The headgear connection features 151, 153 could be any suitable features that are co-operable with the headgear 140 to connect the headgear 140 to the mask body. For example, the headgear connection features 151, 153 could be buckles as shown, or alternatively could be frictional couplers, domes, straps, or any other suitable features.

The headgear 140 comprises an upper headstrap 141 connectable to the upper headgear connection features 151 and a lower headstrap 143 connectable to the lower headgear connection features 153. The upper and lower headstraps 141, 143 are adapted to extend around the back of the patient's head in use

In an alternative configuration (not shown), the headgear 140 comprises a first side strap that is connectable to one of the two upper headgear connection features 151 and connectable to one of the two lower headgear connection features 153 and a second side strap that is connectable to the other of the two upper headgear connection features 151 and connectable to other of the two lower headgear connection features 153. The first and second side straps are configured to loop around the ears of the patient in use.

The headgear 140 may be adjustable. For example, the lengths of the straps may be adjustable at the headgear connection features 151, 153, or may be adjustable along the straps themselves.

The mask may be disposable and may be replaced, either partially or fully, at shorter intervals than the nasal cannula.

A second configuration mask 200 is shown in FIG. 6. Unless described as otherwise below, the features, functionality, and options are the same as the first configuration mask 100, and like reference numbers indicate like parts with the addition of 100.

The mask 200 comprises a frame 211. The mask body 201 is integral with or releasably coupled to the frame 211.

The frame may be sewn, heat welded, ultrasonically welded or overmolded to the mask body.

The frame 211 comprises a pre-formed shape that conforms to the shape of the patient's face.

In the configuration shown, the frame 211 comprises a hollow generally annular shape, having an upper frame portion 211a contoured to match generally to an upper nose region of a patient, an opposite bottom frame portion 211b configured to extend across a patient's chin, and two side frame portions 211c that extend between the ends of the upper frame portion 211a and the lower frame portion 211b. A central opening of the frame 211 is defined between the upper frame portion 211a, lower frame portion 211b, and two side frame portions 211c.

The filter 203, or the mask body 201 and filter 203, extends over the central opening of the frame to filter fluid from the patient facing side of the mask 200 to the non-patient facing side of the mask 200.

The frame 211 may be rigid or at least more rigid than the mask body 201 and/or filter 203.

The mask 200 comprises a cushion 215 configured to seal about or against the patient's face. The seal provided by the cushion 215 against the patient's face may be a full seal, or may alternatively be a partial seal that still enables the flow of gases from the patient facing side of the mask to the non-patient facing side of the mask.

The cushion 215 extends rearwardly and outwardly from the frame 211 and, like the frame 211, comprises a hollow generally annular shape, having an upper cushion portion 215a contoured to match generally to an upper nose region of a patient, an opposite bottom cushion portion 215b configured to extend across a patient's chin, and two side cushion portions 215c that extend between the ends of the upper cushion portion 215a and the lower cushion portion 215b. A central opening of the cushion 215 is defined between the upper cushion portion 215a, lower cushion portion 215b, and two side cushion portions 215c. The frame 211 is on the front of the cushion 215, and the mask body 201 and/or filter 203 is on the front of the frame 211. The frame 211 could be in front of or behind the mask body 201 and/or filter 203.

The mask 200 is selectively securable to or removable from the patient independently of the nasal cannula 10100, 10100′.

The interfacing feature 221 comprises a recess or slot in the cushion 215, wherein the recess or slot conforms to an external surface of the nasal cannula 10100, 10100′ such that the cushion substantially seals about the side arms 10108, 10108′, 10109, 10109′ and/or patient conduit 10300, 10300′ of the nasal cannula.

The cushion 215 may be made from any suitable material, such as silicone for example.

In the configuration shown, the recess of the interfacing feature 221 comprises a key-hole shape recess. The recess has a narrow recess opening 221a and a wide recess cavity 221b. The recess cavity 221b is configured to receive the side arms 10108, 10108′, 10109, 10109′ and/or patient conduit 10300, 10300′. The recess opening 221a width is smaller than the corresponding dimension of the side arm and/or patient conduit so that the cushion 215 needs to be deformed to enter the side arm and/or patient conduit into the recess, and to assist with retaining the cushion 215 in engagement with the side arm and/or patient conduit and to at least partly seal against the side arm and/or patient conduit.

The recess of the interfacing feature 221 extends from a surface 215d of the cushion 215 that contacts the patient's face in use to a portion of the cushion distal from the patient's face.

The recess of the interfacing feature 221 of the mask 200 can be provided in one or both sides 215c of the cushion 215.

The headgear connection features 251, 253 are provided on the frame 211 rather than on the mask body 201 or filter 203.

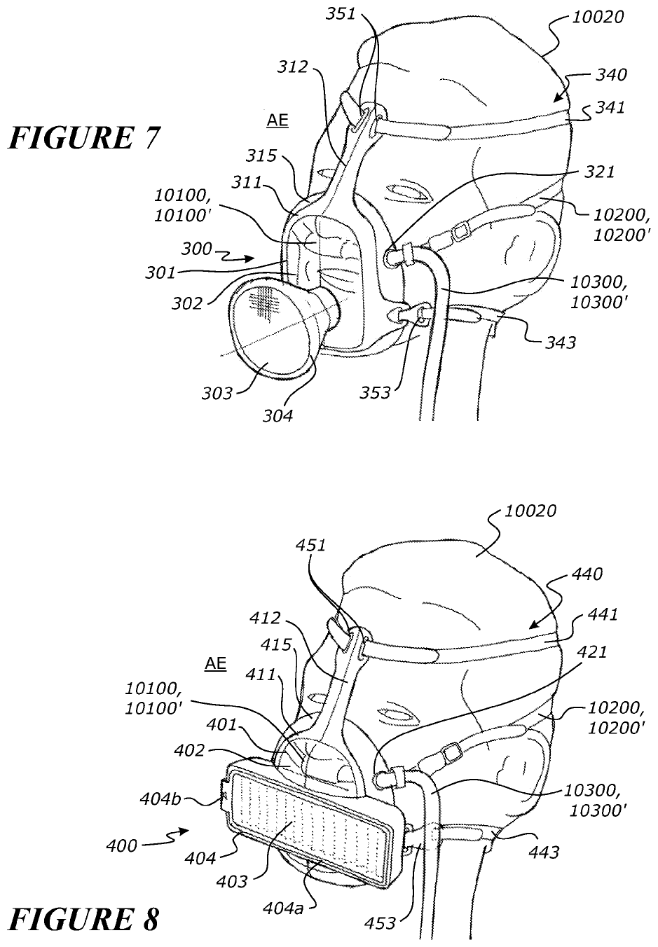

A third configuration mask 300 is shown in FIG. 7. The third configuration mask is a variant of the second configuration mask 200. Unless described as otherwise below, the features, functionality, and options are the same as the second configuration mask 200, and like reference numbers indicate like parts with the addition of 100.

The mask body 301 is configured to define a cavity or part thereof with the patient's face in use. The cavity is configured to at least accommodate a portion of the opening of the patient's airway.

In the configuration shown, the cushion 315, the frame 311, and the mask body 301 together define the cavity with the patient's face in use.

In the configuration shown, the cavity is adapted to accommodate the patient's nose and mouth in use.

In this configuration, the mask body 301 comprises a filter housing 304. The filter housing 304 has a frustoconical configuration extending forward from the mask body, with the narrower portion of the filter housing proximal to the mask body 301 and the wider portion of the filter housing distal from the mask body 301. The filter housing could, alternatively, have any suitable alternative shape such as that shown in FIG. 8 for example.

The filter housing 304, and thereby the filter 303, is arranged at a lower portion of the mask 300 as shown. In the configuration shown, the filter housing 304, and thereby the filter 303, is arranged to be proximal the patient's mouth in use.

The filter 303 is in the filter housing 304. The interior of the filter housing 304 is in fluid communication with the cavity to enable fluid flow from the cavity into the filter 303 in the filter housing 304.

The filter 303 may comprise a planar or flat filter, or alternatively may comprise a pleated, corrugated, or concertina configuration filter to increase the surface area of the filter.

The filter 303 or filter housing 304 may be removable and replaceable. For example, the filter housing 304 containing the filter 303 may be removable from the mask body 301 and replaceable with another filter housing 304 containing a fresh filter 303.

The filter housing 304 connection to the mask body 301 may be an industry standard connection, such as a 22 mm taper connection for example, or may be a proprietary connection.

The mask 300 may comprise a moisture indicator configured to provide an indication of the moisture in the filter 303. The moisture indicator may comprise a hydrochromatic material or coating that changes colour when a predetermined moisture level is reached, to indicate when the filter 303 should be changed. For example, the filter 303 or filter housing 304 may comprise a hydrochromatic material or coating.

In the configuration shown, the mask body 301 comprises a transparent portion 302 to permit a view of a portion of the patient's face in use. The transparent portion 302 is arranged at an upper portion of the mask to permit a view of the patient's nares and the nasal cannula during use. The transparent portion 302 provides a window that is advantageously configured to enable a medical professional to check that the nasal cannula 10100, 10100′ is in place in the patient's nares while using the mask.

In an alternative configuration, the portion 302 of the mask body 301 may not be transparent.

The transparent portion 302 may be hydrophobic to repel moisture to help prevent fogging of the transparent portion. For example, the transparent portion may comprise a hydrophobic material or may be treated to be hydrophobic. Suitable materials comprise one or more polycarbonate, polypropylene, or transparent nylon for example.

Because the filter housing 304 extends forwardly from the mask body 301, that may place extra loading on the headgear 340 of the mask 300. The upper headstrap 341 may be placed higher around the patient's head to resist the loading from the filter housing 304 and filter 303. An upper portion 312 of the frame extends upwardly from the mask body 301 and cushion 315 to a position corresponding to a patient's forehead in use. The upper headgear connection features or forehead support 351 are provided at an upper end of the upper portion 312 of the frame 311. The upper headstrap 341 can extend generally horizontally from the upper headgear connection features 351 around the back of the patient's head. The lower headstrap 343 can extend generally horizontally from the lower headgear connection features 353 around the back of the patient's head or neck.

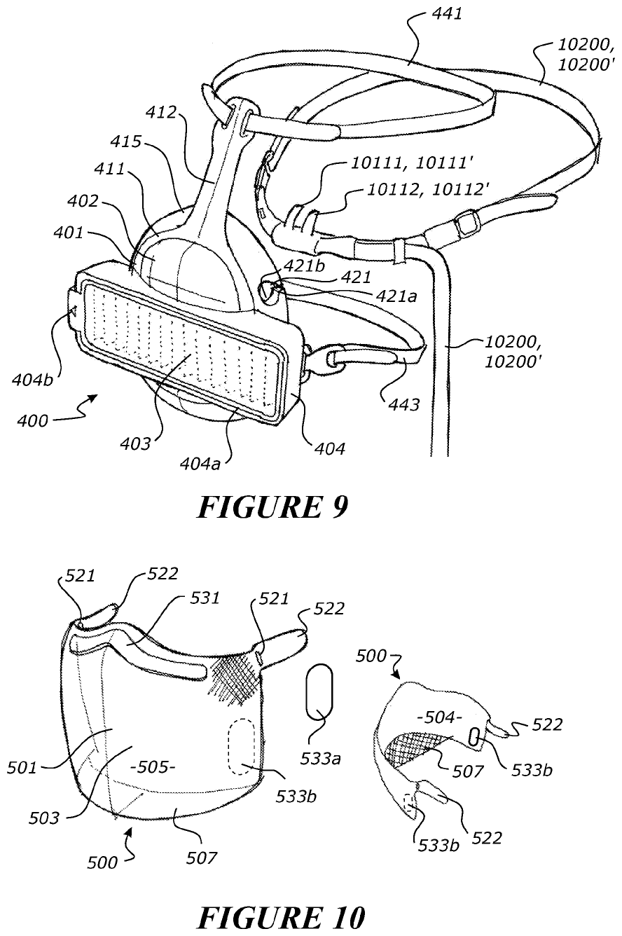

A fourth configuration mask 400 is shown in FIGS. 8 and 9. The fourth configuration mask is a variant of the third configuration mask 300. Unless described as otherwise below, the features, functionality, and options are the same as the third configuration mask 300, and like reference numbers indicate like parts with the addition of 100.

In this configuration, the mask body 401 comprises a filter housing 404 having a different configuration. The filter housing 404 is configured to accommodate a larger filter than that of FIG. 7.

A width of the filter housing 404 is such that it extends transversely beyond sides of the frame 411 and cushion 415 of the mask 400.

The filter housing 404 is a generally rectangular configuration having a wide width dimension and a small height dimension.

The filter housing 404, and thereby the filter 403, is arranged at a lower portion of the mask 400 as shown. In the configuration shown, the filter housing 404, and thereby the filter 403, is arranged to be proximal the patient's mouth in use.

The filter 403 is in the filter housing 404. The interior of the filter housing is in fluid communication with the cavity to enable fluid flow from the cavity into the filter 403 in the filter housing.

The filter 403 may comprise a planar or flat filter, or alternatively may comprise a pleated, corrugated, or concertina configuration filter to increase the filtration surface area of the filter.

The filter 403 is removable from the mask 400.

The mask 400 may comprise a moisture indicator configured to provide an indication of the moisture in the filter 403. The moisture indicator may comprise a hydrochromatic material or coating that changes colour when a predetermined moisture level is reached, to indicate when the filter 403 should be changed. For example, the filter 403 or filter housing 404 may comprise a hydrochromatic material or coating.

In the configuration shown, the filter 403 is removable from the filter housing 404. A section of the filter housing 404 may be removable to provide access to the filter 403 so it can be removed from the filter housing 404 and replaced. For example, a forward section 404a may comprise a hollow frame that holds the filter 403 in position in the filter housing, and a grasping portion 404b may be provided on the forward section 404a to enable a user to remove the forward section 404a and thereby the filter 403.

In the configuration shown, the mask body 401 comprises a transparent portion 402 to permit a view of a portion of the patient's face in use. The transparent portion 402 is arranged at an upper portion of the mask to permit a view of the patient's nose during use. The transparent portion 402 provides a window that is advantageously configured to enable a medical professional to check that the nasal cannula 10100, 10100′ is in place in the patient's nose while using the mask.

A fifth configuration mask 500 is shown in FIG. 10. Unless described as otherwise below, the features, functionality, and options are the same as the first configuration mask 100, and like reference numbers indicate like parts with the addition of 400.

The mask 500 comprises a deformable structure that can be shaped to follow a contour of the patient's face.

In the configuration shown, the mask body 501 comprises the deformable structure 531. The deformable structure 501 is adapted to be manipulated by a user to form a desirable shape.

The deformable structure may be a plastically deformable structure. The deformable structure may comprise a malleable structure that can be bent to provide the desired shape, and will then hold that shape. The deformable structure may comprise one or more bendable, pliable, ductile, and/or pliant materials.

The deformable structure can be formed of any suitable deformable material but may, for example, comprise a metal wire or metal strip. The deformable structure may be overmoulded into the mask body 501. Alternatively, the deformable material may be a deformable polymeric material.

The deformable structure 531 is provided at a top portion of the mask body. In the form shown the deformable structure 531 is at or proximal to the patient's nasal bridge when in use.

The mask 500 is provided with an attachment mechanism configured to releasably attach the mask 500 to the patient's face so that a mask headgear is not used. The attachment mechanism assists with retaining the mask in position on the patient's face in use. The attachment mechanism comprises a first portion 533a configured to adhere to the patient's face and a second portion 533b provided on the mask, the first and second portions adapted to releasably couple with one another.

In the form shown, the attachment mechanism comprises pads 533b on either side the mask. The pads 533b may comprise either hooks or loops that are configured to engage with complementary pads 533a that are adhered to the patient's face and that have the other of the hooks and loops. The pads 533b on the mask and the pads 533a on the patient's face together form a hook-and-loop fastener system. In an alternative configuration, the patient adhering pads 533a may attach directly to the material of the mask body 501, eliminating the pads 533b.

The interfacing mechanism comprises an aperture 521 through which a portion of the nasal cannula is adapted to extend. The interfacing mechanism may comprise two apertures 521. The apertures 521 may be provided one on either side of the mask 500.

The aperture(s) 521 is/are provided on a tab or tabs 522 extending from a side or sides of the mask. The tabs 522 may extend rearwardly from the mask body 501, or may extend from the mask body 501 at a different orientation.

The side arm(s) 10108, 10109, 10108′, 10109′ extend through the aperture(s) 521 of the mask in use.

The conduit/tube 10300, 10300′ can extend between a rear edge of the mask body 501 and the patient's cheek in use.

In use, a buckle and frame component of the nasal cannula may ‘sandwich’ the tab 522 of the mask. For example, for cannula 10100′, the male buckle clip details on the cannula arm(s) 10108′, 10109′ outside side arm mount 10283 may be inserted through aperture(s) 521 before being connected to buckle(s) 13200. As another example, the strap 10200 of cannula 10100 and ends of the side arms 10108, 10109 may be inserted through the aperture(s) 521 before the strap is coupled at buckle 10250.

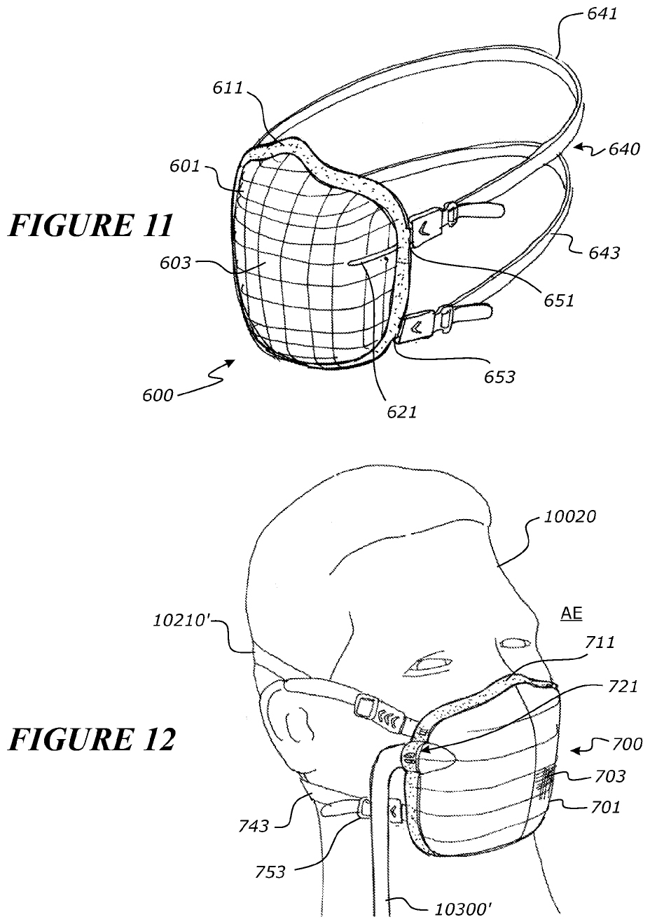

A sixth configuration mask 600 is shown in FIG. 11. Unless described as otherwise below, the features, functionality, and options are the same as the second configuration mask 200, and like reference numbers indicate like parts with the addition of 400.

In this configuration, the mask 600 comprises a frame. The mask body 601 is integral with or releasably coupled to the frame 611.

The mask 600 may or may not comprise a cushion.

In some configurations, the frame 611 is sewn, heat welded, ultrasonically welded or overmolded to the mask body 601.

The frame 611 is provided behind the mask body 601 and/or filter 603. The frame 611 comprises a pre-formed shape that conforms to the shape of the patient's face. The frame 611 could be in front of or behind the mask body 601 and/or filter 603.

The frame 611 may be rigid or at least more rigid than the mask body 601 and/or filter 603. That is, the frame is at least semi-structural, but may have sufficient compliance to provide a comfortable contact against the patient's face.

The upper and lower headgear connection features 651, 653 may be moulded into the frame 611.

The interfacing feature 621 comprises an aperture provided in the mask body 601 to allow a portion of the nasal cannula 10100, 10100′ to extend through the aperture. In the configuration shown, the aperture 621 allows the patient conduit 10300, 10300′ of the nasal cannula to extend through the aperture. Additionally, or alternatively, the aperture 621 could allow one of the side arms 10108, 10108′, 10109, 10109′ of the nasal cannula 10100, 10100′ to extend through the aperture.

The mask may comprise two apertures 621, one in either side of the mask body 601.

The mask body 601 and/or filter 603 may be hydrophilic, may be permeable to water molecules and gases flow, and may be formed into a three dimensional shape.

The material may be substantially impermeable to bulk flow of liquid water.

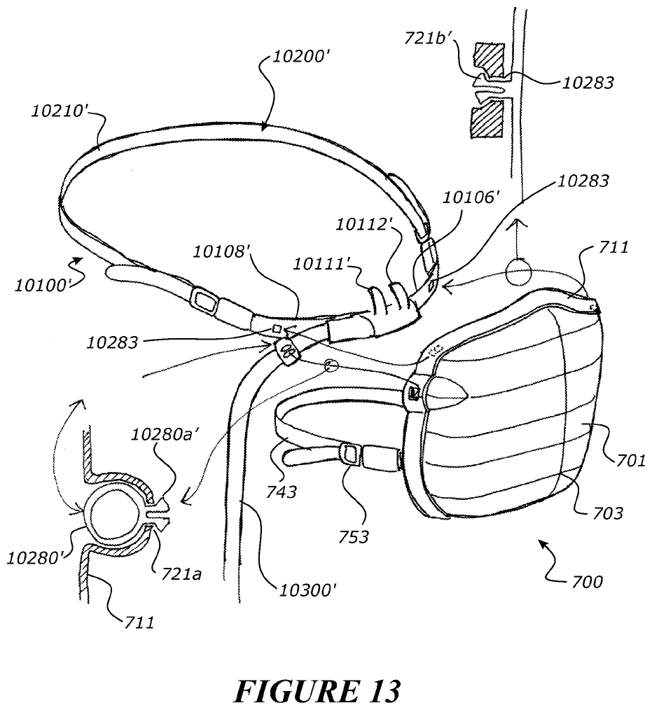

A seventh configuration mask 700 is shown in FIGS. 12 and 13. Unless described as otherwise below, the features, functionality, and options are the same as the sixth configuration mask 600, and like reference numbers indicate like parts with the addition of 100.

The mask 700 is configured to interface with the second configuration non-sealing nasal cannula 10100′.

The mask comprises a frame 711 shaped to conform to an external surface of the patient conduit 10300′ of the nasal cannula 10100′.

The interfacing feature 721 comprises a first mounting structure 721a to releasably couple the frame 711 to the patient conduit 10300′.

The first mounting structure comprises a frame mount 721a adapted to engage with a tube clip lug 10280a′ of a tube clip 10280′ of the patient conduit 10300′. The frame mount 721a may, for example, comprise an aperture or recess in the frame 711. The frame may comprise two frame mounts 721a, one on either side of the frame.

The interfacing feature 721 comprises a second mounting structure 721b to releasably couple the frame to a side arm 10108′, 10109′ of the nasal cannula 10100′.

The second mounting structure 721b comprises a frame lug 721b′ on the mask, and at least one side arm 10108′, 10109′ of the nasal cannula comprises a side arm mount 10283, the frame lug 721b′ adapted to engage with the side arm mount 10283. Alternatively, the configuration could be reversed so that the lug is provided on the cannula frame and the mount is provided on the mask frame 711. The nasal cannula may comprise two side arm mounts 10283, one on either side arm, and the frame 711 may comprise two complementary frame lugs 721b′, one on either side of the frame 711.

The lug(s) 10280a′, 721b′ may comprise resilient spaced apart lug portions and positive engagement features such as barbs or projections, to enable the lug portions to be flexed towards each other during engagement of the components and to assist with avoiding undesirable disengagement of components.

With this configuration, the tube clip 10280′ can be removed from the side arm 10108′, 10109′ of the nasal cannula, and can be attached to the frame mount 721a of the frame 711 of the mask 700. The semi-structural frame 711 of the mask attaches into the side arm mounts 10283 on the side arms 10108′, 10109′ of the nasal cannula 10100′.

The mask headgear may have a single, lower headstrap 743, but the engagement between the mask frame 711 and the nasal cannula 10100′ enable the headstrap 10200′ of the nasal cannula to act as an upper headstrap for the mask 700 in use.

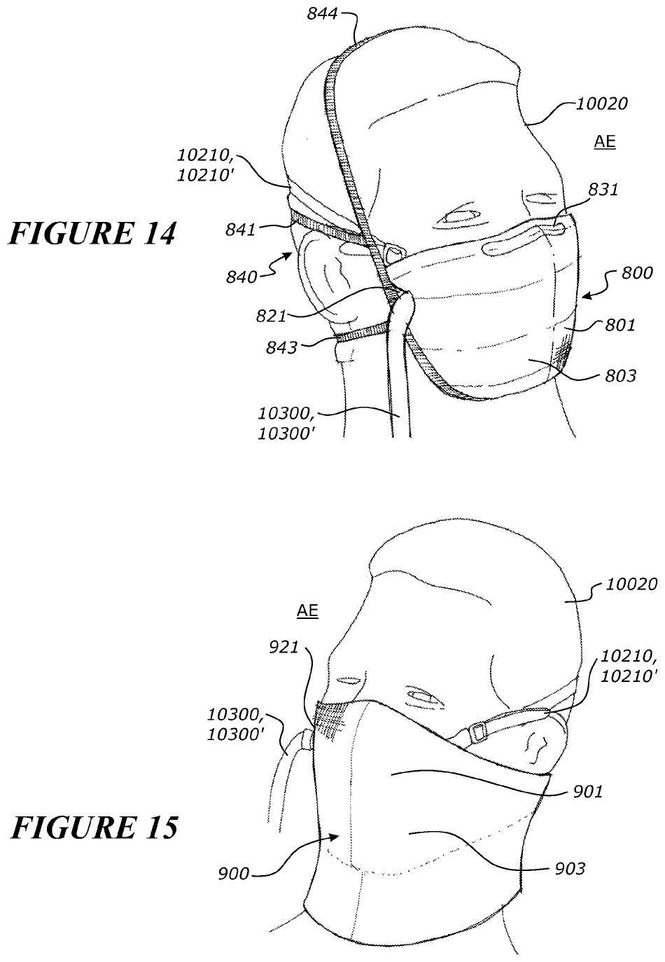

An eighth configuration mask 800 is shown in FIG. 14. Unless described as otherwise below, the features, functionality, and options are the same as the first configuration mask 100, and like reference numbers indicate like parts with the addition of 700.

The mask 800 comprises a deformable structure 831 that can be shaped to follow a contour of the patient's face.

In the configuration shown, the mask body 801 comprises the deformable structure 831. The deformable structure 801 is adapted to be manipulated by a user to form a desirable shape.

The deformable structure 801 may be a plastically deformable structure. The deformable structure 801 may comprise a malleable structure that can be bent to provide the desired shape, and will then hold that shape. The deformable structure 801 may comprise one or more bendable, pliable, ductile, and/or pliant materials.

The deformable structure 831 can be formed of any suitable deformable material but may, for example, comprise a metal wire or metal strip. Alternatively, the deformable material may be a deformable polymeric material.

The deformable structure 831 is provided at a top portion of the mask body. In the form shown the deformable structure 831 is at or proximal to the patient's nasal bridge when in use.

The mask 800 has a mask headgear 840 of the type described above in relation to embodiment 100. The headgear comprises one or more straps coupled to a periphery of the mask body 801. In the configuration shown, the mask headgear 840 comprises an upper headstrap 841 and a lower headstrap 843. The upper and lower headstrap 841, 843 may have adjustable lengths. The mask 800 may have removable headstrap buckles.

The mask headgear 840 additionally has a crownstrap 844. The crownstrap 844 is configured to extend over the top of a patient's head in use. The crownstrap 844 may be adjustable.

The crownstrap 844 extends upwardly from a rear periphery of the mask body 801. In the form shown, the crownstrap 844 extends around a base of the mask at the rear periphery thereof, and up the other side of the mask to form a continuous loop around the mask and over the patient's head. Alternatively, the crownstrap 844 may terminate higher up the mask body 801.

The mask headgear 840 may alternatively only have the crownstrap 844 and one headstrap 841, 843, or alternatively may only have the crownstrap 844 and no headstraps.

The interfacing feature 821 comprises an aperture provided between at least one of the straps and the mask body 801, the aperture configured such that a portion of the nasal cannula 10100, 10100′ can extend through the aperture.

In the form shown, the aperture 821 is provided between the crownstrap 844 and the periphery of the mask body 801.

The aperture 821 allows the patient conduit 10300, 10300′ of the nasal cannula to extend through the aperture. Additionally, or alternatively, the aperture 821 could allow one of the side arms 10108, 10108′, 10109, 10109′ of the nasal cannula 10100, 10100′ to extend through the aperture.

The aperture 821 may be provided between one side of the mask body 801 and the strap. Alternatively, two apertures 821 may be provided between the mask body 801 and the strap, one on either side of the body.

A ninth configuration mask 900 is shown in FIG. 15. Unless described as otherwise below, the features, functionality, and options are the same as the first configuration mask 100, and like reference numbers indicate like parts with the addition of 800.

The mask 900 is configured to extend around a back of the patient's head, and does not comprise a headgear. This can be achieved by having the mask body 901 extend around the back of the patient's head to completely surround a front portion, side portions, and back portion of the patient's head.

In this configuration, the mask body 901 may be formed substantially entirely of the filter 903. Alternatively, a front portion of the mask body 901 may be formed of the filter 903, and the sides and rear of the mask body may be formed of an elastic sheet material.

The mask 900 is configured to extend downwardly from the patient's face towards the patient's chest in use. Alternatively, the mask 900 may terminate approximately at or just below the patient's chin in use.

This mask 900 is a non-sealing mask. That is, the lower region of the mask 900 is open. However, the coverage of the mask is such that it will still adequately filter fluid from the patient facing side to the non-patient facing side of the mask and will limit the travel of aerosols and pathogens.

A retention arrangement may be provided on one side or a rear of the mask 600, to couple side edges or rear edges of the mask together to retain the mask in place around the patient's head. This retention arrangement may comprise fastener(s), clip(s), buckle(s), or the like. In one configuration, the retention arrangement comprises a hook-and-loop fastener system.

The mask will comprise one or more interfacing features 921 of the type described herein to interface with the patient interface. In one configuration, the mask body 901 comprises an aperture or slot to interface with the patient interface. Interfacing features may be provided in both sides of the mask body 901.

A tenth configuration mask 1000 is shown in FIG. 16. Unless described as otherwise below, the features, functionality, and options are the same as the ninth configuration mask 900, and like reference numbers indicate like parts with the addition of 100.

The mask 1000 comprises a deformable structure 1031 that can be shaped to follow a contour of the patient's face.

In the configuration shown, the mask body 1001 comprises the deformable structure 1031. The deformable structure 1031 is adapted to be manipulated by a user to form a desirable shape.

The deformable structure 1031 may be a plastically deformable structure. The deformable structure 1031 may comprise a malleable structure that can be bent to provide the desired shape, and will then hold that shape. The deformable structure 1031 may comprise one or more bendable, pliable, ductile, and/or pliant materials.

The deformable structure 1031 can be formed of any suitable deformable material but may, for example, comprise a metal wire or metal strip. Alternatively, the deformable material may be a deformable polymeric material.

The deformable structure 1031 is provided at a top portion of the mask body. In the form shown the deformable structure 1031 is at or proximal to the patient's nasal bridge when in use.

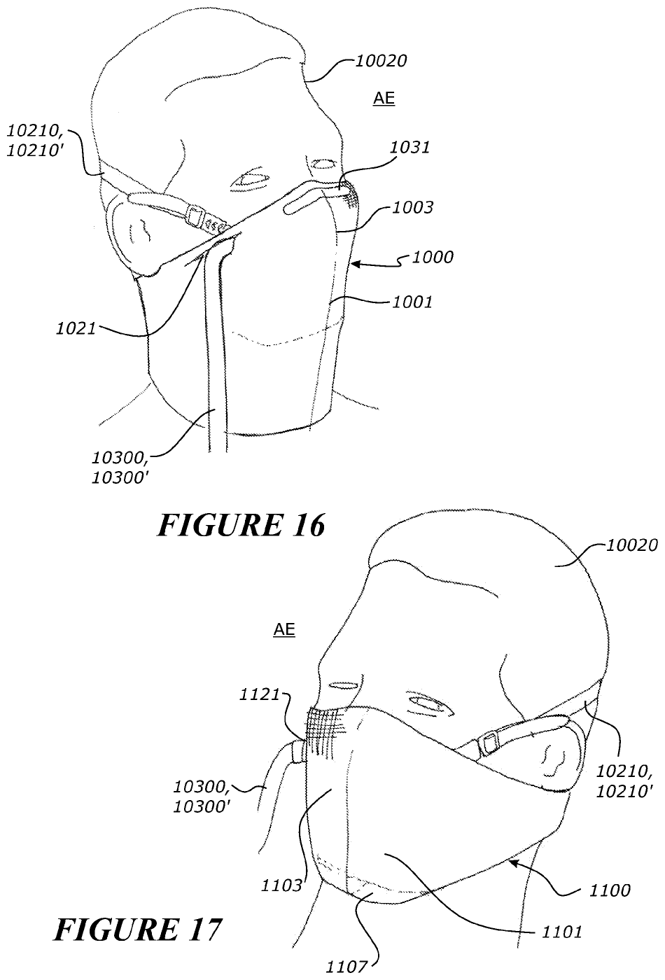

An eleventh configuration mask 1100 is shown in FIG. 17. Unless described as otherwise below, the features, functionality, and options are the same as the ninth configuration mask 900, and like reference numbers indicate like parts with the addition of 200.

The mask body 1101 has a chin portion 1107 that extends across the bottom of the mask body 1101 and under a patient's chin in use. The chin portion 1107 may be formed of the filter 1103 or may alternatively be formed of a different material, such as an elastic material for example. The chin portion 1107 may be a sewn-in elastic fabric or may be formed from the same material as the rest of the mask body.

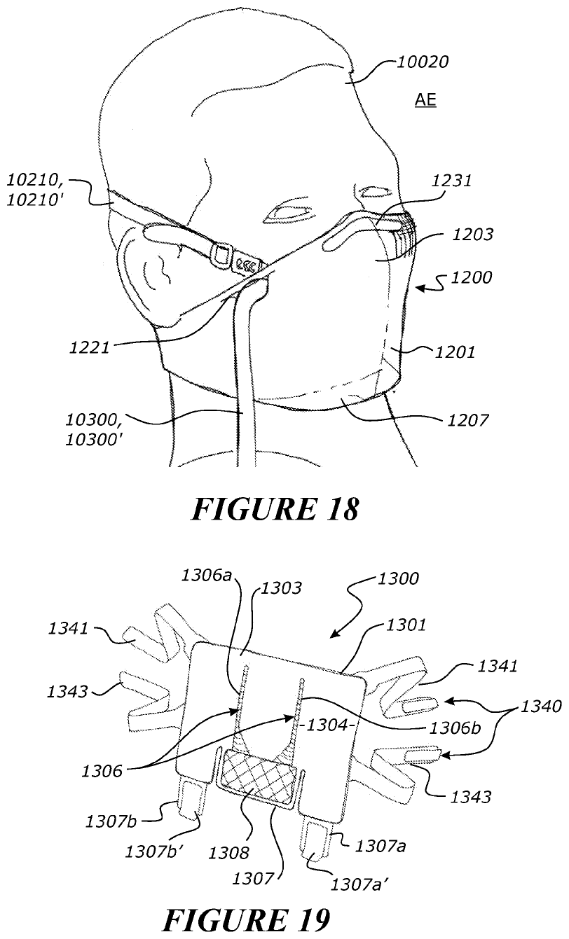

A twelfth configuration mask 1200 is shown in FIG. 18. Unless described as otherwise below, the features, functionality, and options are the same as the tenth configuration mask 1000, and like reference numbers indicate like parts with the addition of 200.

The mask body 1201 has a chin portion 1207 that extends across the bottom of the mask body 1201 and under a patient's chin in use. The chin portion 1207 may be formed of the filter 1203 or may alternatively be formed of a different material, such as an elastic material for example. The chin portion 1207 may be a sewn-in elastic fabric or may be formed from the same material as the rest of the mask body.

A thirteenth configuration mask is shown in FIGS. 19 to 21. Unless described as otherwise below, the features, functionality, and options are the same as the first configuration mask 100, and like reference numbers indicate like parts with the addition of 1200.

A mask 1300 is configured to substantially surround an opening of a patient's airway. The mask comprises a mask body 1301 comprising a filter 1303 configured to enable a flow of gases from a patient facing side of the mask to a non-patient facing side of the mask in use and configured to limit travel of a liquid from the patient facing side 1304 of the mask to the non-patient facing side 1305 of the mask and capture the liquid, to retain liquid on the patient facing side of the mask. The non-patient facing side 1305 of the mask faces an ambient environment AE.

The mask body comprises a transmission arrangement 1306 to transfer the liquid from the filter 1303 to a reservoir 1308.

The mask body 1301 and/or filter 1303 may comprise a pre-formed shape. For example, to generally follow the contours of a patient's face. In some configurations, the mask body and/or filter is rigid or semi-rigid. Alternatively, the filter 1303 may be provided on an underlying material that imparts a pre-formed shape to the filter.

The mask body 1301 is configured to substantially cover a front portion of the patient's face. The mask body 1301 is optionally configured to substantially cover side portions of the patient's face.

The mask body 1301 has a chin portion 1307 that extends across the bottom of the mask body 1301 and under a patient's chin in use. The chin portion 1307 may partly be formed of the filter 1303 or may alternatively be formed of a different material, such as an elastic material for example. The chin portion 1307 may be a sewn-in elastic fabric or may be formed from the same material as the rest of the mask body.

The filter 1303 is configured to limit travel of a liquid from the patient-facing side 1304 of the mask to the non-patient facing side 1305 of the mask. For example, if fluid expelled from a patient's airway contains gas and a liquid, the filter 1303 will limit travel of the liquid from the patient-facing side 1304 of the mask to the non-patient facing side 1305.

The filter 1303 may comprise a material that is permeable to water molecules and gases flow.

The material may be substantially impermeable to bulk flow of liquid water.

Therefore, the filter 1303 may allow the passage of vapour through the mask from the patient facing side 1304 to the non-patient facing side 1305. The filter may absorb liquid from the vapour and/or may allow controlled passage of liquid from the vapour for evaporation from the non-patient facing side 1305 of the mask 1300. The filter 1303 may filter pathogens and/or contaminants from the fluid passing through the filter 1303.

The mask 1300 may comprise an absorbent material to absorb the liquid from the fluid expelled from the patient's airway. The absorbent material can capture the aerosols from the patient, e.g. when a patient coughs and sneezes, and will advantageously prevent droplets from accumulating within the mask.

The filter 1303 may comprise the absorbent material. Alternatively, the absorbent material may be in addition to the filter 1303.

The absorbent material may be hydrophilic. For example, the absorbent material may comprise a hydrophilic material or alternatively may comprise a material that has been treated to be hydrophilic.

In one configuration, the absorbent material is configured to wick the liquid away from the patient. In an alternative configuration, the absorbent material is configured to permit evaporation of the liquid away from the patient, optionally to the ambient environment.

The absorbent material may comprise any suitable material such as, for example, one or more of polyester, polyurethane, nylon, polyethylene or a composite thereof.

The reservoir 1308 may comprise an absorbent pad as shown. The absorbent pad may comprise the absorbent material. Alternatively, the reservoir 1308 may be some other type of receptacle that can receive and retain liquid.

The reservoir 1308 is arranged to be positioned under or proximal to the patient's chin during use. To that end, in the configuration shown the reservoir 1308 is provided on an inner surface of the chin portion 1307 of the mask body 1301.

The reservoir 1308 will assist with providing comfort to the patient by keeping the patient side 1304 of the mask relatively dry while still capturing the moisture on the patient side 1304. This may help reduce irritation of the patient's skin.

The transmission arrangement 1306 maybe integral with the filter 1303 or may be formed separately and coupled to the filter 1303.

In some configurations, the filter 1303 comprises a first absorbent material and the transmission arrangement 1306 comprises a second absorbent material. That is, the filter 1303 and the transmission arrangement 1306 may both be absorbent. The first absorbent material and the second absorbent material may be the same or may be different. Alternatively, the first absorbent material and the second absorbent material may be different or have different properties.

In some configurations, the second absorbent material (of the transmission arrangement 1306) is more absorbent than the first absorbent material (of the filter 1303). The second absorbent material may be selected so as to be more absorbent than the first absorbent material. Alternatively, the second absorbent material may comprise a thickness greater than a thickness of the first absorbent material.

The second absorbent material is shaped to facilitate transfer and movement of the liquid from the filter 1303 to the reservoir 1308. In the form shown, the transmission arrangement comprises one, two, or more columns or channels 1306a, 1306b of the second absorbent material, to enable excess liquid to gravity feed and/or travel by capillary action to the reservoir 1308.

The column(s) or channel(s) 1306a, 1306b may extend substantially vertically when the mask 1300 is in use or, alternatively, may be on some other angle above horizontal, such as at least about 45 degrees, at least about 60 degrees, at least about 70 degrees, or at least about 80 degrees.

The filter may comprise a fabric, such as a textile fabric and/or a polymer matrix fabric for example. Suitable materials comprise one or more of polyester, polyurethane, nylon, or surface-modified polypropylene, natural fibres, cotton, wool, hemp, and/or bamboo for example.

In some configurations, the mask body 1301 and/or filter 1303 may comprise an elastic material, to enable the mask body and/or filter to flex to differently sized or shaped patient's faces.

In some configurations, the mask body 1301 and/or filter 1303 is/are configured to minimize impediment on flow rates of a gases flow through the mask body and/or filter. This enables the mask 1300 to be used in high flow therapy without substantially increasing pressure in the mask to avoid any possibility of lung damage from pressure build-up, while still allowing the high flow to flush the patient's lungs. In some configurations, the mask body 1301 and/or filter 103 is configured to allow a flow rate of a gases flow through it of up to about 450 L/min. In some configurations, the mask body 1301 and/or filter 1303 is configured to allow a flow rate of a gases flow through the mask body and/or filter of about 10-450/min.

The filter 1303 may be porous to allow air to pass through it, to avoid undesirable increases of pressure inside the mask 1300. Open-to-closed ratios of the filter material may be selected to achieve a desired level of porosity. Additionally, or alternatively, the mask body may comprise arrangements of closed portion(s) and filter portion(s) to provide an overall desired level of porosity of the mask.

The mask 1300 covers at least part of the patient interface 10100, 10100′ in use. As outlined above, the patient interface 10100, 10100′ may be a non-sealing patient interface.

The patient interface 10100, 10100′ comprises a non-sealing nasal cannula comprising one or more prongs 10111, 10112, 10111′, 10112′ that extend(s) into the patient's nasal passage(s) in use, side arms 10108, 10108′, 10109, 10109′ that extend laterally along the patient's face in use, and a patient conduit 10300, 10300′ adapted to connect to a gases source for delivering a gases flow to the patient via the prong(s).

In the configuration shown, the mask 1300 is adapted to cover the nose and mouth of the patient.

The mask 1300 does not comprise a gases supply or gases removal conduit and/or does not comprise a connection port for connecting to a gas source or gas removal device and/or is not adapted to actively deliver a flow of gases to the patient and/or is not adapted to actively remove a flow of gases from the patient. That is, gases flow is delivered to the patient via the patient interface 10100, 10100′ rather than via the mask 1300. The purpose of the mask 1300 is to filter fluids from the patient rather than delivering gases to the patient.

The mask 1300 comprises a moisture indicator 1310 configured to provide an indication of the moisture in the filter. The moisture indicator comprises a hydrochromatic material or coating that changes colour when a predetermined moisture level is reached. This can provide a visual indication of when a maximum moisture level in the filter 1303 is reached.

The moisture indicator 1310 is provided on a front, non-patient facing surface of the mask body so that it is readily viewable by a medical professional.

The mask 1300 comprises an upper portion adapted to cover the patient's nose and/or mouth in use, and a lower portion adapted to position under the patient's chin in use. The upper portion comprises the upper portion of the mask body 1301. The lower portion comprises the chin portion 1307.

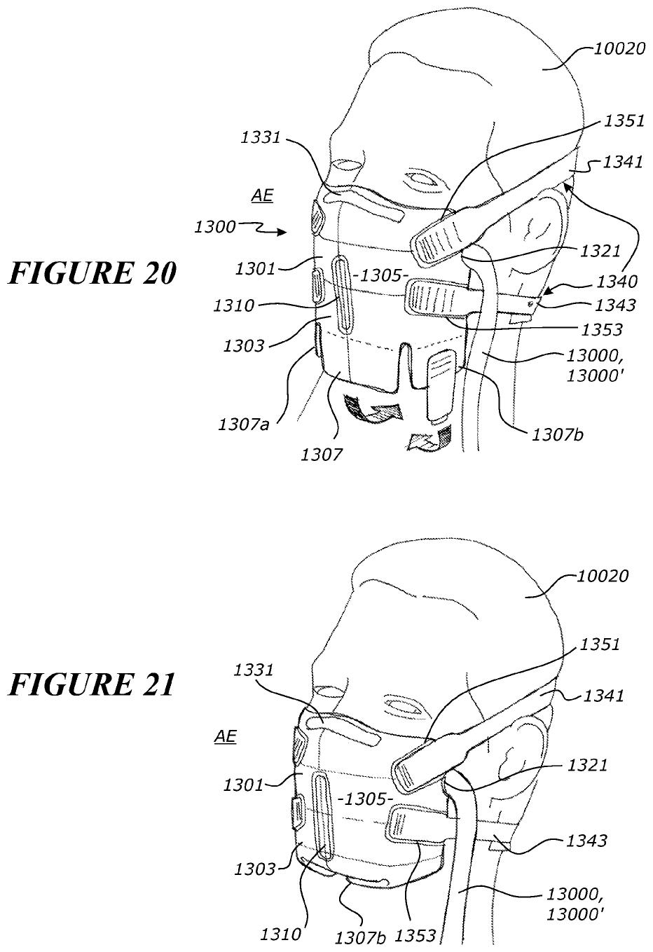

In the configuration shown, the lower portion comprises discontinuous regions or under chin strap portions 1307, 1307a, 1307b that are configured to overlap and couple with one another to form a chin strap. The discontinuous regions can be moved in the directions of the arrows in FIG. 20 by first moving portion 1307 under the patient's chin and then moving the portions 1307a, 1307b under portion 1307.

At least some of the discontinuous regions 1307, 1307a, 1307b comprise a fastening mechanism 1307a′, 1307b′ to couple said discontinuous regions together. The fastening mechanism may comprise clips, fasteners, an adhesive, or a hook-and-loop fastener system for example.

The mask 1300 comprises a headgear 1340 and/or headgear connection features to secure the mask to the patient independently of the nasal cannula 10100, 10100′. The headgear 1340 may be integral with the mask 1300 or may be releasably connectable to the headgear connection features.

The headgear could be any of the types described for the embodiments above.

In the configuration shown, the headgear 1340 comprises two upper headstraps 1341 and two lower headstraps 1343. The upper headstraps 1341 and the lower headstraps 1343 are connectable to each other via fastening mechanisms. The fastening mechanism may comprise clips, fasteners, an adhesive, or a hook-and-loop fastener system for example.

In the configuration shown, the mask comprises a pair of upper headgear connection features 1351, one on either side of the mask body 1301 at or adjacent a rear edge of the mask body. The mask further comprises a pair of lower headgear connection features 1353, one on either side of the mask body 1301 at or adjacent a rear edge of the mask body.

The mask may comprise one or more interfacing features 1321 configured to, in use, interface with a patient interface provided on the patient 10020. The interfacing feature may allow a portion of the nasal cannula 10100, 10100′ to extend therethrough. For example, the interfacing feature may allow the patient conduit 10300, 10300′ of the nasal cannula and/or may allow one of the side arms 10108, 10108′, 10109, 10109′ of the nasal cannula 10100, 10100′ to extend through the interfacing feature. Alternatively, a portion of the patient cannula may extend between a rear peripheral edge of the mask body 1301 and the patient's face in use.

The mask 1300 comprises a deformable structure 1331 that can be shaped to follow a contour of the patient's face.

In the configuration shown, the mask body 1301 comprises the deformable structure 1331. The deformable structure 1301 is adapted to be manipulated by a user to form a desirable shape.

The deformable structure 1331 may be a plastically deformable structure. The deformable structure 1331 may comprise a malleable structure that can be bent to provide the desired shape, and will then hold that shape. The deformable structure 1331 may comprise one or more bendable, pliable, ductile, and/or pliant materials.

The deformable structure 1331 can be formed of any suitable deformable material but may, for example, comprise a metal wire or metal strip. Alternatively, the deformable material may be a deformable polymeric material.

The deformable structure 1331 is provided at a top portion of the mask body 1301. In the form shown the deformable structure 1331 is at or proximal to the patient's nasal bridge when in use.

Alternatively, the mask body 1301 may not have the deformable structure 1331.

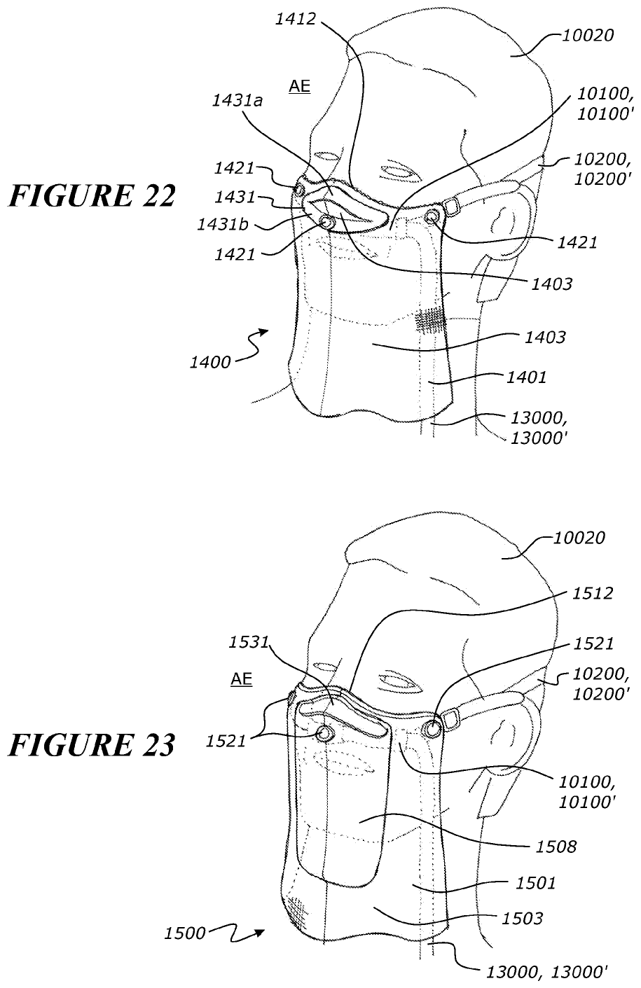

A fourteenth configuration mask 1400 is shown in FIG. 22. Unless described as otherwise below, the features, functionality, and options are the same as the tenth configuration mask 1000, and like reference numbers indicate like parts with the addition of 400.

The mask 1400 comprises a deformable structure 1431 that can be shaped to follow a contour of the patient's face.

The interfacing feature 1421 comprises at least one mounting mechanism to couple the mask to the nasal cannula 10100, 10100′.

The mounting mechanism of the interfacing feature 1421 may, for example, comprise one or more fasteners mounted to the mask that are engageable with complementary features on the nasal cannula 10100, 10100′. The fasteners may be clips or snap fasteners for example. In the form shown, the fasteners comprise dome snaps that are releasably engageable with complementary dome snaps on the nasal cannula.

The mounting mechanism of the interfacing feature 1421 enables the mask body 1401 to be removably attached to the nasal cannula 10100, 10100′. The mask body 1401 can be removed from the nasal cannula, discarded, and replaced.

The mask 1400 may comprise one of said mounting mechanisms. Alternatively, the mask 1400 may comprise a plurality of the mounting mechanisms.

In the configuration shown, a central one of the mounting mechanisms 1421 is mounted to part of the deformable structure 1431 or alternatively to a frame member of pre-formed shape, to provide support to the central one of the mounting mechanisms 1421. The central one of the mounting mechanisms 1421 is engageable with the face mount part 10110, 10110′ of the nasal cannula.

In the configuration shown, the deformable structure 1431 comprises an upper portion 1431a that is at or proximal to the patient's nasal bridge when in use. The deformable structure 1431 comprises a lower portion 1431b that has a central portion spaced from the upper portion 1431b, and that is positioned beneath the patient's nose in use in the region of the patient's upper lip. The deformable structure 1431 can be shaped to follow a contour of a patient's face. The central one of the mounting mechanisms 1421 is coupled to the lower portion 1431b. Alternatively, the deformable structure 1431 may comprise a single strip.

Outer ones of the mounting mechanisms 1421 are mounted to the mask body 1401 at or adjacent a rear peripheral edge of the mask body. The outer mounting mechanism are engageable with the side arms 10108, 10108′, 10109, 10109′ of the nasal cannula.

An edge of the mask 1400 comprises a padded and/or absorbent material 1412. In the configuration shown, the padded and/or absorbent material 1412 extends across an upper edge of the mask body 1401 on the rear surface thereof, to provide a comfortable contact between the upper edge of the mask and the patient's face. The padded and/or absorbent material may be configured to form at least a partial seal with the patient's face.

The absorbent material may be any of the types of absorbent material described herein.

A fifteenth configuration mask 1500 is shown in FIG. 23. Unless described as otherwise below, the features, functionality, and options are the same as the fourteenth configuration mask 1400, and like reference numbers indicate like parts with the addition of 100.

In this configuration, a forward portion of the mask body 1501 comprises an absorbent pad 1508. The absorbent pad 1508 may comprise an absorbent material to absorb the liquid from the fluid expelled from the patient's airway. The absorbent material can capture the aerosols from the patient, e.g. when a patient coughs and sneezes, and will advantageously prevent droplets from accumulating on a surface within the mask.

The absorbent material may be hydrophilic. For example, the absorbent material may comprise a hydrophilic material or alternatively may comprise a material that has been treated to be hydrophilic.

In one configuration, the absorbent material is configured to wick the liquid away from the patient. In an alternative configuration, the absorbent material is configured to permit evaporation of the liquid away from the patient, optionally to the ambient environment.

The absorbent material may comprise any suitable material such as, for example, one or more of polyester, polyurethane, nylon, polyethylene or a composite thereof.

The absorbent pad 1508 may extend over at least a major part of the front of the mask body 1501. In one configuration, the absorbent pad is configured to extend from the deformable structure 1531 to at least the chin of a patient.

The mask 1500 may comprise a moisture indicator configured to provide an indication of the moisture in the filter 1503 or absorbent pad 1508. The moisture indicator may comprise a hydrochromatic material or coating that changes colour when a predetermined moisture level is reached, to indicate when the mask should be changed. For example, the filter 1503 or absorbent pad 1508 may comprise a hydrochromatic material or coating.

In the configuration shown, the deformable structure 1531 comprises a single strip that is at or proximal to the patient's nasal bridge when in use. Alternatively, the deformable structure 1531 could have a configuration similar to that of the fourteenth configuration.

The central one of the mounting mechanisms 1521 is mounted to the absorbent material 1508 rather than to the deformable structure 1531. The central one of the mounting mechanisms 1521 could alternatively be mounted to a different part of the mask body 1501.

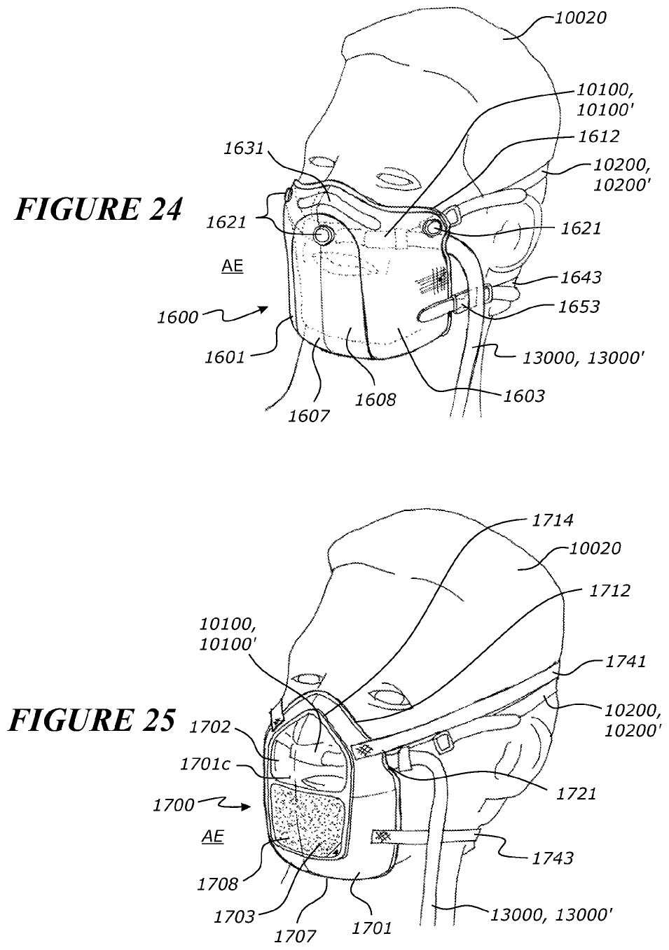

A sixteenth configuration mask 1600 is shown in FIG. 24. Unless described as otherwise below, the features, functionality, and options are the same as the fifteenth configuration mask 1500, and like reference numbers indicate like parts with the addition of 100.

In this configuration, the mask body 1601 is shaped to conform to the shape of the patient's face, and includes a chin portion 1607 that extends across the bottom of the mask body 1601 and under a patient's chin in use. The chin portion 1607 may be formed of the filter 1603 or may alternatively be formed of a different material, such as an elastic material for example. The chin portion may be a sewn-in elastic fabric or may be formed from the same material as the rest of the mask body.

An edge of the mask body 1601 comprises a padded and/or absorbent material 1612. In the configuration shown, the padded and/or absorbent material 1612 extends across an upper edge of the mask body 1601, down the sides of the mask body, and along the chin portion on the rear surface thereof, to provide a comfortable contact between the upper edge of the mask and the patient's face. The padded and/or absorbent material may be configured to form at least a partial seal with the patient's face around the perimeter of the mask.

The mask may comprise one or more headgear connection features 1653 that is attachable to a headstrap of the patient interface, or that is attachable or attached to a mask headstrap 1643.

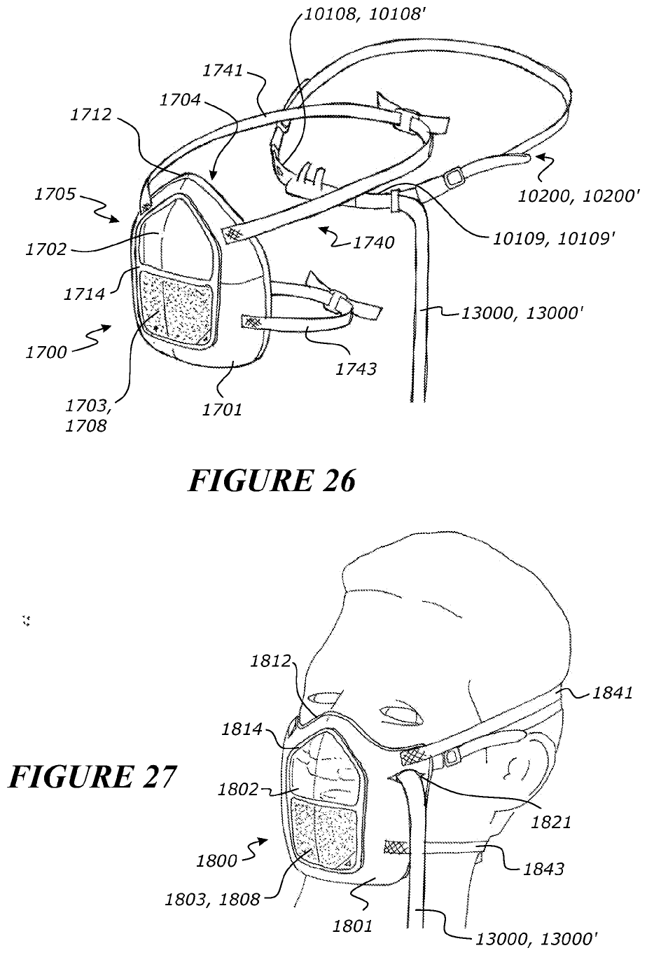

A seventeenth configuration mask 1700 is shown in FIGS. 25 and 26. Unless described as otherwise below, the features, functionality, and options are the same as the third configuration mask 300, and like reference numbers indicate like parts with the addition of 1400.

The mask 1700 is configured to substantially surround an opening of a patient's airway. The mask 1700 comprises a mask body 1701 configured to define a cavity 1701c or part thereof with the patient's face in use, the cavity 1701c configured to at least accommodate a portion of the opening of the patient's airway.

The mask body 1701 is configured to enable a flow of gases from a patient facing side of the mask to a non-patient facing side of the mask in use.

The mask body comprises a transparent portion 1702 to permit a view of a portion of the patient's face in use.

The mask 1700 comprises an absorbent material 1708 configured to absorb moisture within the cavity during use. The absorbent material 1708 may be provided in an absorbent pad as described for the configurations above.

The cavity 1701c is adapted to accommodate the patient's nose and mouth in use.

The mask 1700 covers at least part of the patient interface 10100, 10100′ in use. As outlined above, the patient interface 10100, 10100′ may be a non-sealing patient interface. The patient interface may be a non-sealing nasal cannula 10100, 10100′.

The transparent portion 1702 is arranged at an upper portion of the mask to permit a view of the patient's nose during use. The transparent portion 1702 provides a window that is advantageously configured to enable a medical professional to check that the nasal cannula 10100, 10100′ is in place in the patient's nose while using the mask.

The transparent portion 1702 may be hydrophobic to repel moisture to help prevent fogging of the transparent portion. For example, the transparent portion may comprise a hydrophobic material or may be treated to be hydrophobic. Suitable materials comprise one or more polycarbonate, polypropylene, or transparent nylon for example.

The mask body 1701 may comprise a filter 1703 configured to filter a fluid from a patient facing side 1704 of the mask to a non-patient facing side 1705 of the mask. The non-patient facing side 1705 of the mask faces an ambient environment AE.

The mask body 1701 is shaped to conform to the shape of the patient's face, and includes a chin portion 1707 that extends across the bottom of the mask body 1601 and under a patient's chin in use. The chin portion 1707 may be formed of the filter 1703 or may alternatively be formed of a different material, such as an elastic material for example. The chin portion may be a sewn-in elastic fabric or may be formed from the same material as the rest of the mask body.

The mask body 1701 is configured to substantially cover a front portion of the patient's face. The mask body 1701 is optionally configured to substantially cover side portions and chin portion of the patient's face.

The filter 1703 is configured to limit travel of a liquid from the patient-facing side 1704 of the mask to the non-patient facing side 1705 of the mask. For example, if fluid expelled from a patient's airway contains gas and a liquid, the filter 1703 will limit travel of the liquid from the patient-facing side 1704 of the mask to the non-patient facing side.

The filter 1703 may comprise a material that is permeable to water molecules and gases flow.

The material may be substantially impermeable to bulk flow of liquid water.

Therefore, the filter 1703 may allow the passage of vapour through the mask from the patient facing side 1704 to the non-patient facing side 1705. The filter may absorb liquid from the vapour and/or may allow controlled passage of liquid from the vapour for evaporation from the non-patient facing side 1705 of the mask 1700. The filter 1703 may filter pathogens and/or contaminants from the fluid passing through the filter 1703. The absorbent material 1708 is configured to absorb the liquid from the fluid expelled from the patient's airway. The absorbent material can capture the aerosols from the patient, e.g. when a patient coughs and sneezes, and will advantageously prevent droplets from accumulating within the mask.

The filter 1703 may comprise the absorbent material 1708. Alternatively, the absorbent material 1708 may be in addition to the filter 1703.

The absorbent material 1708 may be hydrophilic. For example, the absorbent material may comprise a hydrophilic material or alternatively may comprise a material that has been treated to be hydrophilic.

In one configuration, the absorbent material 1708 is configured to wick the liquid away from the patient. In an alternative configuration, the absorbent material is configured to permit evaporation of the liquid away from the patient, optionally to the ambient environment.

The absorbent material 1708 may comprise any suitable material such as, for example, one or more of polyester, polyurethane, nylon, polyethylene or a composite thereof.

The filter 1703 may comprise a fabric, such as a textile fabric and/or a polymer matrix fabric for example. Suitable materials comprise one or more of polyester, polyurethane, nylon, or surface-modified polypropylene, natural fibres, cotton, wool, hemp, and/or bamboo for example.

In some configurations, the mask body 1701 and/or absorbent material 1708 and/or filter 103 is/are configured to minimize impediment on flow rates of a gases flow through the mask body and/or filter. This enables the mask 1700 to be used in high flow therapy without substantially increasing pressure in the mask to avoid any possibility of lung damage from pressure build-up. In some configurations, the mask body 1701 and/or absorbent material 1708 and/or filter 1703 is configured to allow a flow rate of a gases flow through it of up to about 450 L/min. In some configurations, the mask body 1701 and/or absorbent material 1708 and/or filter 1703 is configured to allow a flow rate of a gases flow through the mask body and/or absorbent material and/or filter of about 10-450 L/min.

The filter 1703 may be porous to allow air to allow air to pass through it, to avoid undesirable increases of pressure inside the mask 1700. Open-to-closed ratios of the filter material may be selected to achieve a desired level of porosity. Additionally, or alternatively, the mask body may comprise arrangements of closed portion(s) and filter portion(s) to provide an overall desired level of porosity of the mask.

One or more of the absorbent material 1708, filter 1703, and mask body 1701 is/are formed of or comprises the material that is permeable to water molecules and gases flow and/or hydrophilic material.

The material(s) may be substantially impermeable to bulk flow of liquid water.

The absorbent material 1708 and/or filter 1703 may be provided in a moulded housing component 1714. The moulded housing component 1714 may be shaped to a patient's nasal/facial form.

The absorbent material 1708, filter 1703, and/or housing component 1714 may be removable from the mask and replaceable. For example, the housing component 1714 containing the filter 1703 and absorbent material 1708 may be removable from the filter body 1701 and replaceable.

The absorbent material 1708 may extend from the filter 1703 to a portion of the mask body 1701. The mask body 1701 may comprise a transmission arrangement to transfer the liquid from the filter 1703 to the absorbent material 1708. The transmission arrangement may be of the type described in relation to transmission arrangement 1306 for example.

The filter 1703 is arranged at a lower portion of the mask 100. In the configuration shown, the filter 1703 is arranged to be proximal the patient's mouth in use.

The mask 1700 comprises a moisture indicator configured to provide an indication of the moisture in the filter or the absorbent material. The moisture indicator may comprise a hydrochromatic material or coating that changes colour when a predetermined moisture level is reached, to indicate when the mask should be changed. For example, the filter 1703 or absorbent pad 1708 may comprise a hydrochromatic material or coating.

The absorbent material 1708 may be hydrophilic. For example, the absorbent material may comprise a hydrophilic material or alternatively may comprise a material that has been treated to be hydrophilic.

The mask comprises a seal 1712 configured seal about or against the patient's face. An edge of the mask body comprises the seal 1712.

In the configuration shown, the seal 1712 comprises a strip that extends across an upper edge of the mask body 1701, down the sides of the mask body, and along the chin portion on the rear surface thereof, to provide a comfortable contact between the upper edge of the mask and the patient's face. The seal may be configured to form at least a partial seal with the patient's face around the perimeter of the mask.

A portion of the patient interface such as nasal cannula 10100, 10100′ may extend between a rear peripheral edge of the mask body 1701 and the patient's face in use.

In the form shown, the patient conduit 13000, 13000′ or one or both of the side arms 10108, 10108′, 10109, 10109′ of the nasal cannula may pass or extend through region(s) at the rear perimeter of the mask body 1701. The seal 1712 may flex to enable that passage.

In some configurations, the mask may comprise in interfacing feature 1721 to, in use, interface with the non-sealing patient interface. In the configuration shown, the portion(s) of the seal 1712 that form the interfacing feature 1721 may be more compliant than other portions of the seal, wherein the compliant portion(s) is/are configured to conform to an external portion of the non-sealing patient interface during use.

The interfacing feature 1721 may be provided on one or both sides of the mask body 1701.

The mask 1700 further comprises a headgear 1740 or headgear connection features, which may be of any of the types of headgear or headgear connection features described herein. In the configuration shown, the headgear 1740 comprises an upper headstrap 1741 having ends that connect to an upper portion of the mask body, and a lower headstrap 1743 having ends that connect to a lower portion of the mask body.

The mask 1700 does not comprise a gases supply or gases removal conduit and/or does not comprise a connection port for connecting to a gas source or gas removal device and/or is not adapted to actively deliver a flow of gases to the patient and/or is not adapted to actively remove a flow of gases from the patient.

By providing the disclosed mask 1700 with transparent portion 1702, a medical professional can view the placement of the prongs of the nasal cannula on the patient's face. Due to high humidity involved in nasal high flow and relatively low flow rates out from the mask, there could be a tendency for the transparent portion to fog up. By providing the absorbent material 1708, the likelihood of fogging is reduced or eliminated. The absorbent material can also play a role in aerosol capture, thereby preventing or substantially reducing the spread of any pathogens.

An eighteenth configuration mask 1800 is shown in FIG. 27. Unless described as otherwise below, the features, functionality, and options are the same as the seventeenth configuration mask 1700, and like reference numbers indicate like parts with the addition of 100.

In this configuration, the interfacing feature 1821 comprises an aperture provided in the mask body 1801 to allow a portion of the non-sealing patient interface such as nasal cannula 10100, 10100′ to extend through. In the configuration shown, the aperture 1821 allows the patient conduit 10300, 10300′ of the nasal cannula to extend through the aperture. Additionally, or alternatively, the aperture 1821 could allow one of the side arms 10108, 101018′, 10109, 10109′ of the nasal cannula 10100, 10100′ to extend through the aperture.

The aperture of the interfacing feature 1821 may be provided in a side of the mask body 1801. In some configurations, two of said apertures 1821 may be provided. The two apertures 1821 may be provided in opposing sides of the mask body 1801.

Rather than being an aperture, the interfacing feature 1821 may comprise a recess or a slot.

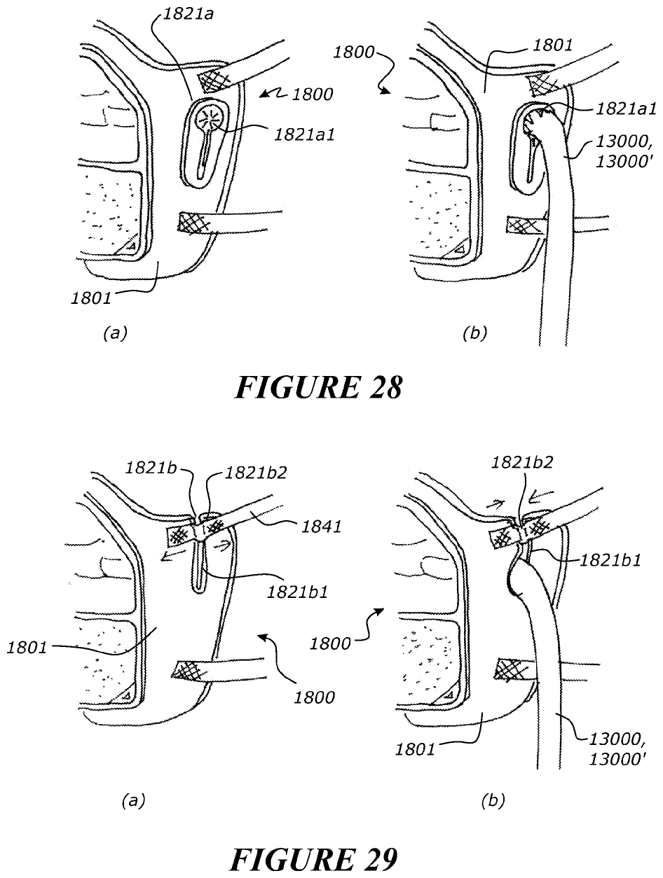

Exemplary interfacing features 1821 are shown in FIGS. 28(a) to 31(b), and labelled 1821a-1821d.

In the configuration of FIGS. 28(a) and 28(b), the interfacing feature 1821a comprises an elastomer with a tearable portion 1821a1 that is sealed when not torn, and that can be torn or broken by pushing a portion of the cannula 10100, 10100′ (such as the side arm 10108, 10109, 10108′, 10109′ or patient conduit 13000, 13000′) through the tear region 1821a. That allows the portion of the patient interface to extend therethrough during use.

In the configuration shown, the tearable portion 1821a1 comprises a slit with an enlarged end. The enlarged end can be any suitable shape, but in the form shown is rounded. The slid enables a wider conduit to be passed through than would otherwise be the case.

In the configuration of FIGS. 29(a) and 29(b), the interfacing feature 1821b comprises a slot 1821b1 that extends into the mask body 1801 from the periphery or the mask body. A strap 1821b2 covers an end of the slot 1821b1 to define an aperture to allow a portion of the patient interface to extend through the aperture in use. The strap 1821b may be a portion of the mask headstrap 1841. The strap 1821b2 may be elastic so it can stretch to enlarge the slot 1821b1 to enable a portion of the cannula such as a connector to be extended therethrough. The elastic strip will then contract to close around at least part of the patient conduit 13000, 13000′.

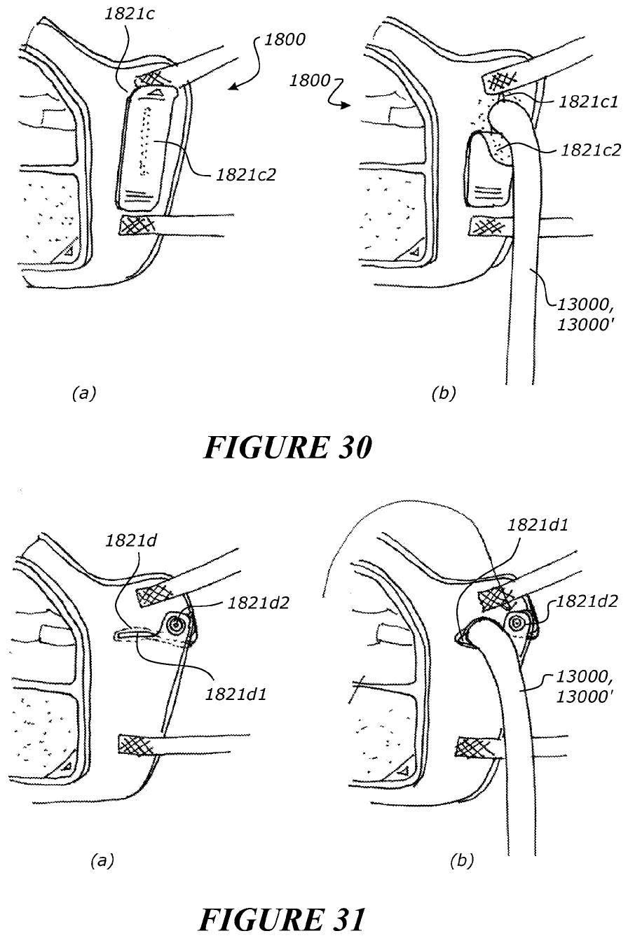

In the configuration of FIGS. 30(a) and 30(b), the interfacing feature 1821c comprises an aperture 1821c1 in the mask body 1801. A cover 1821c2 is at least partly removable from the mask body 1801. The cover 1821c2 is configured to cover the aperture 1821c1 in a first configuration and to expose the aperture 1821c1 in a second configuration. The cover 1821c2 can be removed, or partly removed, from the mask body 1801 to enable a portion of the cannula 10100, 10100′ (such as the side arm or patient conduit) to extend through the aperture 1821c1.

The at least partly removable cover 1821c2 is adapted to couple with a portion of the mask body 1801 via a fastening mechanism, such as via a hook-and-loop fastener system for example.

In the configuration of FIGS. 31(a) and 31(b), the interfacing feature 1821d comprises a recess or slot 1821d1 that extends into the mask body 1801 from its periphery. A portion of the mask body comprises a coupling mechanism 1821d1 that is engageable with an overlapping portion of the mask body, to couple ends of the recess or slot 1821d1 together to define an aperture to allow a portion of the cannula 10100, 10100′ to extend through the aperture during use. The coupling mechanism 1821d1 could be any suitable configuration, such as a fastener or dome snap for example.

The mask 1700 or 1800 may be provided in combination with a non-sealing patient interface such as non-sealing nasal cannula 10100, 10100′ or any other suitable non-sealing patient interface, to provide an interface system.

When the mask comprises a mask headgear 1740, 1840, the cannula headgear 10200, 10200′ and mask headgear 1740, 1840 are separate such that the mask 1700, 1800 is selectively securable to or removable from the patient independently of the non-sealing nasal cannula 10100, 10100′.

In the interface system, gases flow to the patient is only provided by the non-sealing nasal cannula 10100, 10100′ or other non-sealing patient interface. The gases flow provided by the cannula 10100, 100100′ may be humidified. The mask 1700, 1800 does not comprise a gases supply or gases removal conduit and/or does not comprise a connection port for connecting to a gas source or gas removal device and/or is not adapted to actively deliver a flow of gases to the patient and/or is not adapted to actively remove a flow of gases from the patient.

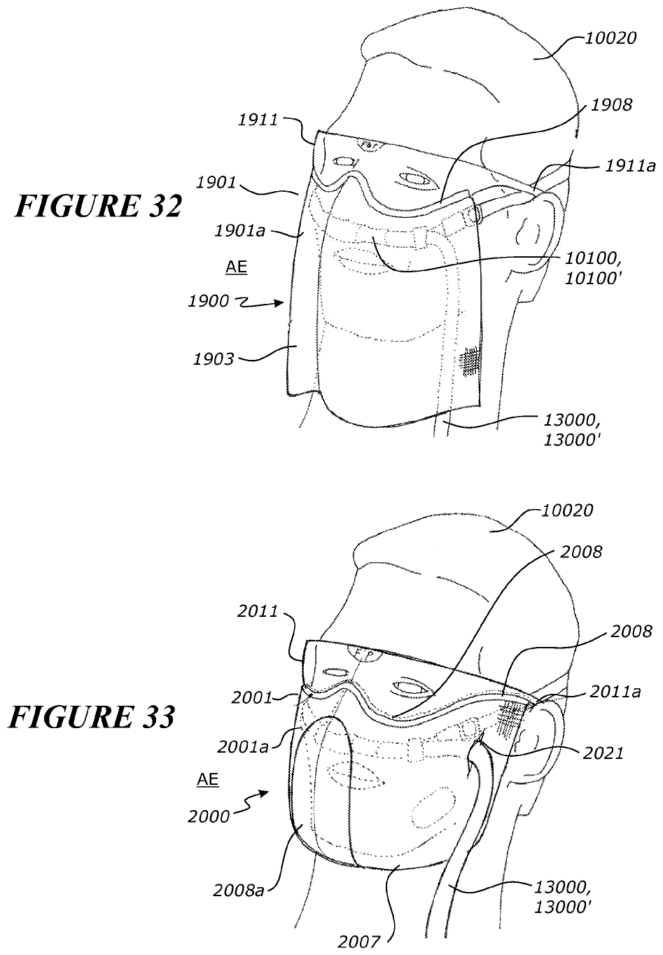

A nineteenth configuration mask 1900 is shown in FIG. 32. Unless described as otherwise below, the features, functionality, and options are the same as the seventeenth configuration mask 1700, and like reference numbers indicate like parts with the addition of 200.

In this configuration, the mask body 1901 comprises a frame 1911 that is configured to position on an upper portion of the patient's face above the patient's nares. A remainder 1901a of the mask body extends from a lower portion of the frame 1911 and is configured to accommodate an opening of the patient's airway in use.

The remainder 1901a of the mask body is configured to accommodate the patient's nose and mouth in use.

The remainder 1901a of the mask body may be integral with or releasably coupled to the frame 1911.

The frame 1911 may be sewn, heat welded, ultrasonically welded or overmolded to the remainder 1901a of the mask body, for example. Alternatively, the remainder 1901a of the mask body may be removably connected to the frame 1911 at the lower portion, optionally via a hook-and-loop fastener system.

When the remainder 1901a of the mask body is removable, it can be removed from the frame 1911, discarded, and replaced.

The remainder 1901a of the mask body may comprise a fabric 1903, such as a textile fabric and/or a polymer matrix fabric for example. The fabric is configured to drape over the patient's face. The side edges and lower edge of the fabric are not secured to the patient.

The fabric 1903 may comprise a filter configured to filter a fluid from a patient facing side of the mask to a non-patient facing side of the mask. The non-patient facing side of the mask faces an ambient environment.

The filter is configured to limit travel of a liquid from the patient-facing side of the mask to the non-patient facing side of the mask. For example, if fluid expelled from a patient's airway contains gas and liquid, the filter will limit travel of the liquid from the patient-facing side of the mask to the non-patient facing side.

The fabric 1903 may comprise a material that is permeable to water molecules and gases flow.

The material may be substantially impermeable to bulk flow of liquid water.

Therefore, the fabric 1903 may allow the passage of vapour through the mask from the patient facing side 1904 to the non-patient facing side 1905. The fabric may absorb liquid from the vapour and/or may allow controlled passage of liquid from the vapour for evaporation from the non-patient facing side 1905 of the mask 1900. The fabric 1903 may filter pathogens and/or contaminants from the fluid passing through the fabric 1903. The fabric 1903 may be hydrophilic. For example, the fabric may comprise a hydrophilic material or alternatively may comprise a material that has been treated to be hydrophilic.

The fabric 1903 may comprise an absorbent material. The absorbent material is configured to absorb the liquid from the fluid expelled from the patient's airway. The absorbent material can capture the aerosols from the patient, e.g. when a patient coughs and sneezes, and will advantageously prevent droplets from accumulating within the mask.

Alternatively, the absorbent material may be in addition to the fabric 1703.

In one configuration, the absorbent material is configured to wick the liquid away from the patient. In an alternative configuration, the absorbent material is configured to permit evaporation of the liquid away from the patient, optionally to the ambient environment.

The absorbent material may comprise any suitable material such as, for example, one or more of polyester, polyurethane, nylon, polyethylene or a composite thereof.

The frame 1911 comprises a pre-formed shape that conforms to the shape of the patient's face.

The frame 1911 is rigid or more rigid than the remainder 1901a of the mask body.

The frame 1911 is configured to extend across the patient's orbital region in use, i.e. to extend across the patient's eyes in use.

The frame 1911 comprises a transparent material. The transparent material can enable the patient to see through the frame and to enable a medical professional to see the patient's eyes in use. The frame 1911 provides the transparent portion of the mask body 1901.

The frame 1911 may be hydrophobic to repel moisture to help prevent fogging of the transparent portion. For example, the frame may comprise a hydrophobic material or may be treated to be hydrophobic. Suitable materials comprise one or more polycarbonate, polypropylene, or transparent nylon for example.

The frame 1911 may be generally eyeglasses-shaped.

The frame 1911 may comprise a retention mechanism to retain the mask 1900 on the patient's head.

In one configuration, the frame 1911 is configured to extend to the sides of the patient's head. In the configuration shown, the frame 1911 is configured to extends towards the back of the patient's ears to retain the mask on the patient's face in use. The frame comprises frame ends 1911a configured to loop behind the patient's ears to secure the mask on the patient's face.

In an alternative configuration, the frame 1911 may be configured to extend to the rear of the patient's head to retain the mask 1900 on the patient's head. The portions of the frame 1911 that extend to the rear of the patient's head may be configured to removably couple to each other.

The frame 1911 comprises a first material 1908 extending along a length of the frame and arranged to be positioned below the patient's eyes in use. In the configuration shown, the first material 1908 is provided at the lower portion of the frame 1911 that the remainder 1901a of the mask body is coupled to.

The first material 1908 may comprise the absorbent material.

The first material 1908 may be in the form of a pad.

The first material 1908 may be configured to seal against the patient's face in use, to form an at least partial seal between the frame 1911 and the patient's face between the frame 1911 and the remainder 1901a of the mask body. This can assist with avoiding humidified gases from fogging up the frame 1911 and/or assist with avoiding high flow of gases from irritating the patient's eyes.

A twentieth configuration mask 2000 is shown in FIG. 33. Unless described as otherwise below, the features, functionality, and options are the same as the nineteenth configuration mask 1900, and like reference numbers indicate like parts with the addition of 100.

In this configuration, the mask body 2001 comprises a pre-formed shape. For example, to generally follow the contours of a patient's face. In some configurations, the mask body is rigid or semi-rigid.

The mask body 2001 also has a chin portion 2007 that extends across the bottom of the mask body 2001 and under a patient's chin in use. The chin portion 2007 may be formed of the fabric 2003 or may alternatively be formed of a different material, such as an elastic material for example. The chin portion may be a sewn-in elastic fabric or may be formed from the same material as the rest of the mask body.

In this configuration, a forward portion of the remainder of the mask body 2001a comprises an absorbent pad 2008a, similar the absorbent pad 1508 of the fifteenth configuration mask. The absorbent pad 2008a may comprise an absorbent material to absorb the liquid from the fluid expelled from the patient's airway. The absorbent material can capture the aerosols from the patient, e.g. when a patient coughs and sneezes, and will advantageously prevent droplets from accumulating within the mask.

The absorbent material may be hydrophilic. For example, the absorbent material may comprise a hydrophilic material or alternatively may comprise a material that has been treated to be hydrophilic.

In one configuration, the absorbent material is configured to wick the liquid away from the patient. In an alternative configuration, the absorbent material is configured to permit evaporation of the liquid away from the patient, optionally to the ambient environment.

The absorbent material may comprise any suitable material such as, for example, one or more of polyester, polyurethane, nylon, polyethylene or a composite thereof.

The absorbent pad 2008a may extend over at least a major part of the front of the remainder of the mask body 2001. In one configuration, the absorbent pad is configured to extend from the patient's nares to at least the chin of the patient.

The mask 2000 may comprise a moisture indicator configured to provide an indication of the moisture in the filter 2003 or absorbent pad 2008a. The moisture indicator may comprise a hydrochromatic material or coating that changes colour when a predetermined moisture level is reached, to indicate when the mask should be changed. For example, the filter 2003 or absorbent pad 2008 may comprise a hydrochromatic material or coating.

The mask body 2001 further comprises an interfacing feature 2021 configured to, in use, interface with a patient interface provided on the patient 10020.

In the form shown, the interfacing feature 2021 comprises an aperture provided in the mask body 2001 to allow a portion of the nasal cannula 10100, 10100′ to extend through. In the configuration shown, the aperture 2021 allows the patient conduit 10300, 10300′ of the nasal cannula to extend through the aperture. Additionally, or alternatively, the aperture 2021 could allow one of the side arms 10108, 10108′, 10109, 10109′ of the nasal cannula 10100, 10100′ to extend through the aperture.

The aperture 2021 may be provided in a side of the mask body 2001. In some configurations two of said apertures 2021 may be provided. The two apertures 2021 may be provided in opposing sides of the mask body 2001.

The interfacing feature(s) 2021 could have any of the alternative configurations described herein.

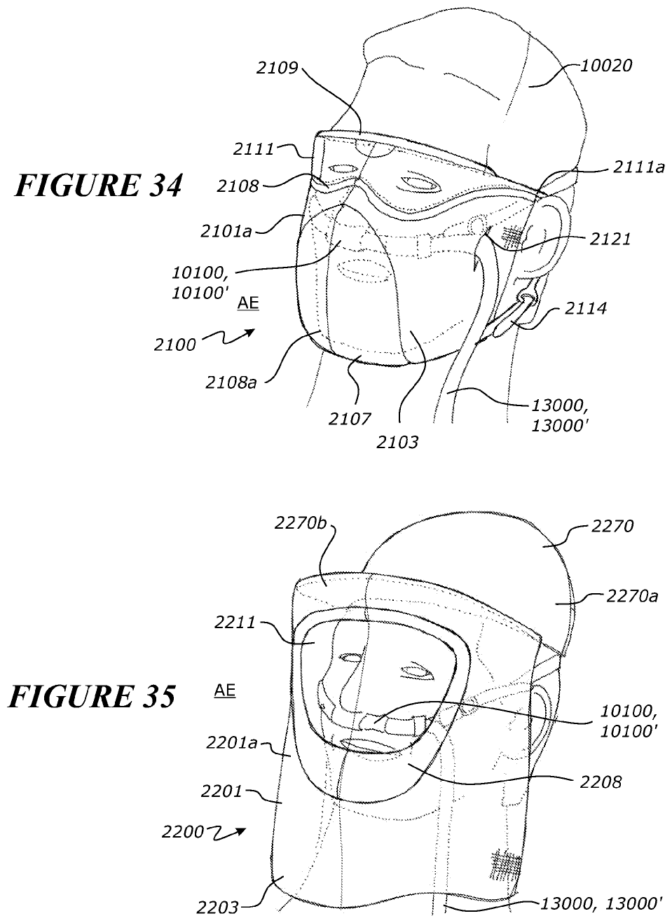

A twenty-first configuration mask 2100 is shown in FIG. 34. Unless described as otherwise below, the features, functionality, and options are the same as the twentieth configuration mask 2000, and like reference numbers indicate like parts with the addition of 100.

In this configuration, the frame 2111 comprises a second material 2109 and arranged to be positioned above the patient's eyes in use.

The second material 2109 extends along a length of the frame 2111.

The second material 2109 may comprise the absorbent material.

The second material 2109 may be in the form of a pad.

The second material 2109 may be configured to seal against the patient's face in use, to form an at least partial seal between the frame 2111 and the patient's face.

The first material 2108 and second material 2109 may comprise the same material, or may be different materials. In some configurations, the first material 2108 and the second material 2109 may connect to form a substantially continuous material periphery around a portion of the patient's face in use; e.g. around the patient's eye region.

The mask 2100 comprises an additional retention mechanism configured to retain the mask on the patient's face in use. The additional retention mechanism comprises a strap 2114 that extends between and connects a bottom edge of the remainder 2101a of the mask body 2101 and the frame end 2011a that is configured to loop behind the patient's ears to secure the mask on the patient's face. A strap 2114 may be provided on each side of the mask. The straps 2114 may be length-adjustable.

A twenty-second configuration mask 2200 is shown in FIG. 35. Unless described as otherwise below, the features, functionality, and options are the same as the twenty-first configuration mask 2100, and like reference numbers indicate like parts with the addition of 100.

In this configuration, the frame 2211 comprises a transparent visor that extends from a region above the patient's nares to a region above, at, or below the patient's nares. The transparent visor 2211 allows a medical professional to see the cannula on the patient and enables the patient to see out of the mask.

A forward portion of the mask body 2201 comprises an absorbent pad 2208. The absorbent pad 2208 extends around a periphery of the frame/visor 2211, i.e. around the top, sides, and bottom of the frame/visor 2211.

The absorbent pad 2208 may comprise an absorbent material to absorb the liquid from the fluid expelled from the patient's airway. The absorbent material can capture the aerosols from the patient, e.g. when a patient coughs and sneezes, and will advantageously prevent droplets from accumulating within the mask.

The absorbent material may be hydrophilic. For example, the absorbent material may comprise a hydrophilic material or alternatively may comprise a material that has been treated to be hydrophilic.

In one configuration, the absorbent material is configured to wick the liquid away from the patient. In an alternative configuration, the absorbent material is configured to permit evaporation of the liquid away from the patient, optionally to the ambient environment.

The absorbent material may comprise any suitable material such as, for example, one or more of polyester, polyurethane, nylon, polyethylene or a composite thereof.

The absorbent pad 2208 may be configured to provide three-dimensional structure to the adjacent region(s) of the remainder 2201a of the mask body.

At least a portion of the frame/visor 2211 may comprise a material that dispels liquids or transmits liquids away from said portion, or said portion of the frame/visor may be treated to dispel liquid or transmit liquid away from said portion. For example, the frame/visor 2211 may be hydrophobic to repel moisture to help prevent fogging of the transparent portion. For example, the frame/visor may comprise a hydrophobic material or may be treated to be hydrophobic. Suitable materials comprise one or more polycarbonate, polypropylene, or transparent nylon for example.

Additionally, or alternatively, the frame/visor is configured to allow liquids to travel to the absorbent pad.

An upper portion of the mask 2200 is configured to couple with a headgear that retains the mask on the patient.

In the configuration shown, the upper portion of the mask above the frame/visor 2211 is configured to couple to a cap 2270. The cap 2270 has a cap body 2270a for fitting to the patient's head, and a projecting front brim 2270b.

The upper portion of the mask may be removably attached to the headgear. For example, the upper portion of the mask may be removably attached to the brim 2270b of the cap or a different region of the cap. The removable attachment may be via a hook-and-loop fastening system for example.

The mask body 2201 can drape from the front brim 2270b of the cap.

The mask 2200 may be provided with a moisture indicator.

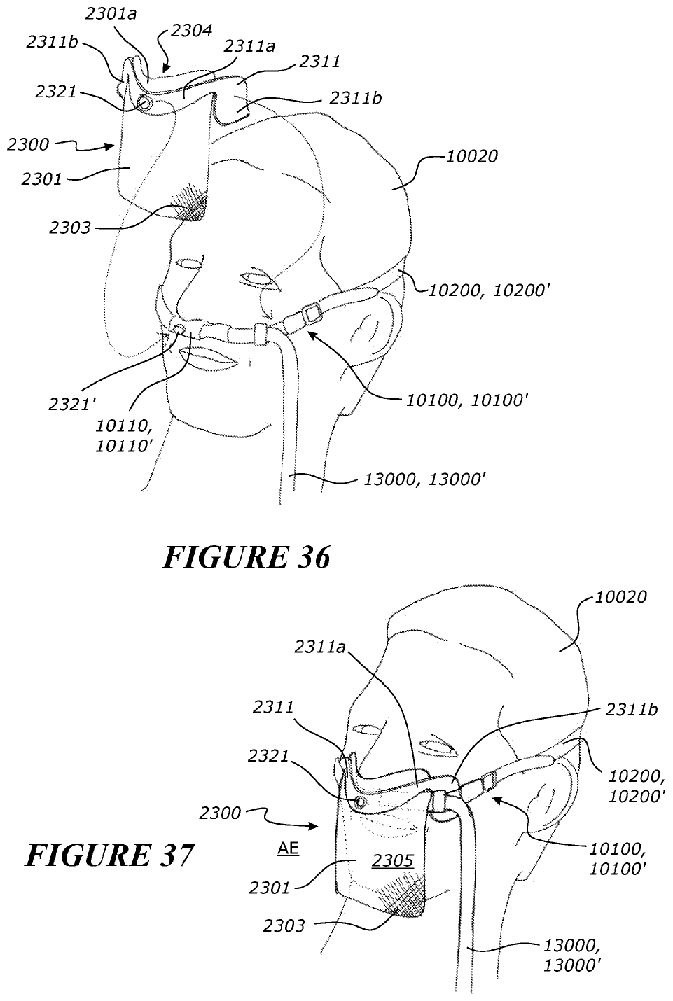

A twenty-third configuration mask 2300 is shown in FIGS. 36 and 37. Unless described as otherwise below, the features, functionality, and options are the same as the fourteenth configuration mask 1400, and like reference numbers indicate like parts with the addition of 900.

The mask 2300 acts as a barrier to cover the nares and mouth of the patient, and part of the non-sealing patient interface such as the prongs of the nasal cannula 10100, 10100′ in use.

The mask 2300 can be provided or used on the non-sealing patient interface.

The mask 2300 comprises a frame 2311. The frame 2311 comprises a transverse member 2311a that extends across an upper region of the mask body 2301. The frame 2311 is configured to extend across a lower portion of the user's nose in use. The frame 2311 may also extend at least partly across the user's cheeks in use.

The frame 2311 may be structural or may be semi-structural to substantially hold its pre-formed shape. Alternatively, the frame 2311 may comprise a deformable structure that can be shaped to follow a contour of the patient's face.

Either side of the frame 2311 comprises a pad 2311b. The pads 2311b extend rearwardly and downwardly from the ends of the transverse member 2311a.

The pads 2311b are configured to sit behind the side arms 10108, 10109, 10108′, 10109′ of the nasal cannula 10100, 10100′. The pads may rest against the patient's cheeks in use.

The interfacing feature 2321 comprises at least one mounting mechanism to couple the mask to the non-sealing patient interface such as the nasal cannula 10100, 10100′ to support the mask on the patient's face.

The mounting mechanism of the interfacing feature may, for example, comprise one or more fasteners mounted to the mask that are engageable with complementary features on the nasal cannula 10100, 10100′. The fastener(s) may be clips or snap fasteners for example. In the form shown, the fastener comprises a dome snap 2321 that is releasably engageable with a complementary dome snap 2321′ on the nasal cannula.

In the configuration shown, the dome snap 2321 is mounted to the transverse member 2311a of the frame 2311, to provide support to the dome snap 2321. The dome snap 2321 is engageable with the complementary dome snap 2321′ on the face mount part 10110, 10110′ or non-contact region 10107 of the nasal cannula 10100, 10100′.

The mounting mechanism of the interfacing feature 2321 enables the mask body 2301 to be removably attached to the nasal cannula 10100, 10100′ to support the mask 2300 on the patient's face. The mask body 2301 can be removed from the nasal cannula, discarded, and replaced.

The mask 2300 may be supported on the patient solely via the non-sealing patient interface such as the nasal cannula 10100, 10100′; i.e. without a mask headgear.

The mask 2300 may comprise one of said mounting mechanisms. Alternatively, the mask 2300 may comprise a plurality of the mounting mechanisms.

The mask body 2301 may be formed substantially entirely of the filter 2303. The filter 2303 may be any of the types or configurations described herein. Alternatively or additionally, the mask body 2301 may comprise an absorbent material. The absorbent material may be any of the types or configurations described herein.

An upper region 2301a of the mask body is positioned above the frame 2311 and may contact the patient's face in use. The upper region 2301a may comprise the filter material and/or absorbent material and/or a cushion material.

The mask body 2301 is configured to extend downwardly from the frame 2311 to terminate approximately at or just below the patient's chin in use. Alternatively, the mask body 2301 may extend from the patient's face to towards the patient's chest in use. By extending at least to a user's chin, a longer length of filter material is provided to allow wicking of moisture down the mask body 2301.

This mask 2300 is a non-sealing mask. That is, the lower region and sides of the mask body 2301 are open. However, the coverage of the mask is such that it will still adequately filter fluid from the patient facing side 2304 to the non-patient facing side 2305 of the mask and will limit the travel of aerosols and pathogens.

The lower region of the mask body 2301 can be lifted up to check the that the prongs of the nasal cannula 10100, 10100′ are properly seated in the patient's nares.

The mask 2300 may comprise a moisture indicator configured to provide an indication of the moisture in the filter 2303. The moisture indicator may comprise a hydrochromatic material or coating that changes colour when a predetermined moisture level is reached, to indicate when the mask should be changed. For example, the filter 2303 may comprise a hydrochromatic material or coating.

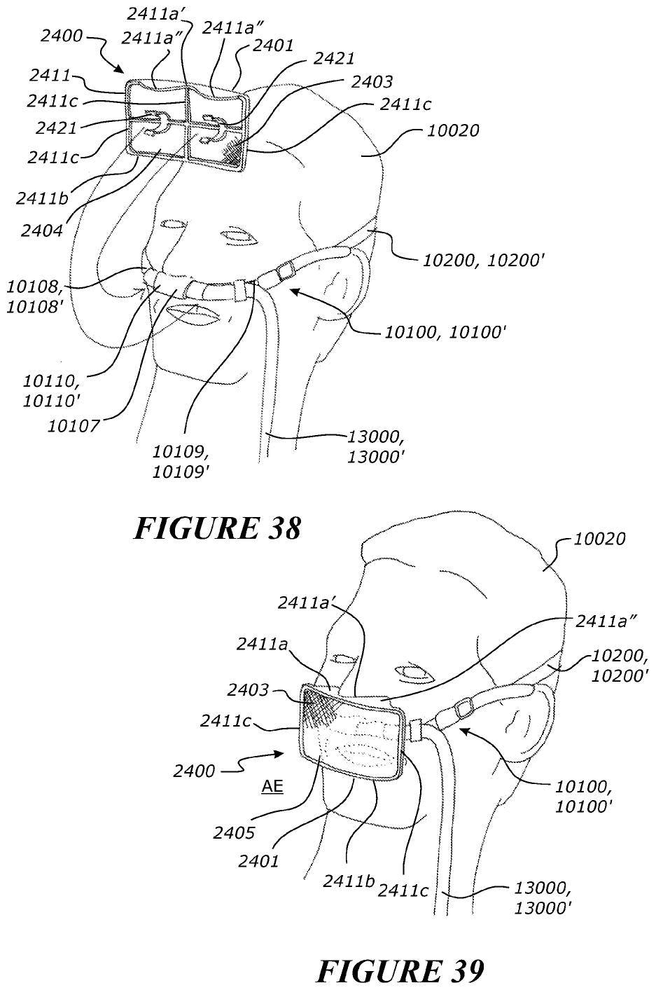

A twenty-fourth configuration mask 2400 is shown in FIGS. 38 and 39. Unless described as otherwise below, the features, functionality, and options are the same as the twenty-third configuration mask 2300, and like reference numbers indicate like parts with the addition of 100.

The mask 2400 acts as a barrier to cover the nares and mouth of the patient, and part of the non-sealing patient interface such as the prongs of the nasal cannula 10100, 10100′ in use.

The mask 2400 can be provided or used on the non-sealing patient interface.

In the configuration shown, the mask 2400 is configured to not substantially cover the patient's chin in use, and covers a smaller region of the patient's face than the twenty-third configuration mask 2300.

The mask body 2401 comprises a frame 2411. The frame 2411 comprises an upper transverse member 2411a that extends across an upper region of the mask body 2401 of the mask 2400, and is configured to extend across a lower portion of the user's nose in use. The upper transverse member 2411a also extends at least partly across the user's cheeks in use.

The upper transverse member 2411a comprises a central recess 2411a′ for receipt of the patients' nose and two projecting regions 2411a″, one on either side of the central recess 2411a′, that are shaped to follow the contours of the patient's face around the nose and inner cheek regions.

The frame 2411 further comprises a lower transverse member 2411b that is configured to be positioned beneath the patient's mouth in use. Two or more upright members 2411c extend between the upper member 2411a and lower member 2411b. The frame may further comprise one or more additional transverse members that extend between the side members 2411c.

The frame 2411 may be structural or may be semi-structural to substantially hold its pre-formed shape. Alternatively, the frame 2411, or part of the frame, may comprise a deformable structure that can be shaped to follow a contour of the patient's face. For example, the upper transverse member 2411a may be deformable to be shaped to the contours of the patient's face.

The interfacing feature 2421 comprises at least one mounting mechanism to couple the mask 2400 to the non-sealing patient interface such as the nasal cannula 10100, 10100′.

The mounting mechanism of the interfacing feature 2421 may, for example, comprise one or more fasteners mounted to the mask that are engageable with complementary features on the nasal cannula 10100, 10100′.

The fastener(s) may be clips or snap fasteners for example. In the form shown, the fasteners comprise clips 2421 on the frame 2411 that are releasably engageable with a feature on the nasal cannula. For example, the clips 2421 may be releasably engageable with the face mount part 10110, 10110′, non-contact region 1017, side arms 10108, 10109, 10108′, 10109′ and/or the patient conduit 13000, 13000′ of the nasal cannula 10100, 10100′. The mask 2400 may have one, two, or more of the clips.

The clip(s) 2421 may have a generally C-shaped configuration, or any other suitable shape to releasably engage with the feature on the nasal cannula.

The clip(s) 2421 may be resilient, and may be an interference fit with the feature on the nasal cannula.

The clip(s) 2421 may extend rearwardly from the frame 2411.

The mounting mechanism of the interfacing feature 2421 enables the mask 2400 to be removably attached to the nasal cannula 10100, 10100′. The mask 2400 can be removed from the nasal cannula, discarded, and replaced.

The mask 2400 may be supported on the patient solely via the non-sealing patient interface such as the nasal cannula 10100, 10100′; i.e. without a mask headgear.

The mask 2400 may comprise one of said mounting mechanisms. Alternatively, the mask 2400 may comprise a plurality of the mounting mechanisms.

The frame 2411 defines one or more apertures that are covered by the filter 2403.

The filter 2403 may be any of the types or configurations described herein. The filter 2403 may an absorbent material. The absorbent material may be any of the types or configurations described herein.

This mask 2400 is a non-sealing mask. That is, the lower region and sides of the mask body 2401 are open. However, the coverage of the mask is such that it will still adequately filter fluid from the patient facing side 2404 to the non-patient facing side 2405 of the mask and will limit the travel of aerosols and pathogens.

The mask 2400 may comprise a moisture indicator configured to provide an indication of the moisture in the filter 2403. The moisture indicator may comprise a hydrochromatic material or coating that changes colour when a predetermined moisture level is reached, to indicate when the mask should be changed. For example, the filter 22403 may comprise a hydrochromatic material or coating.

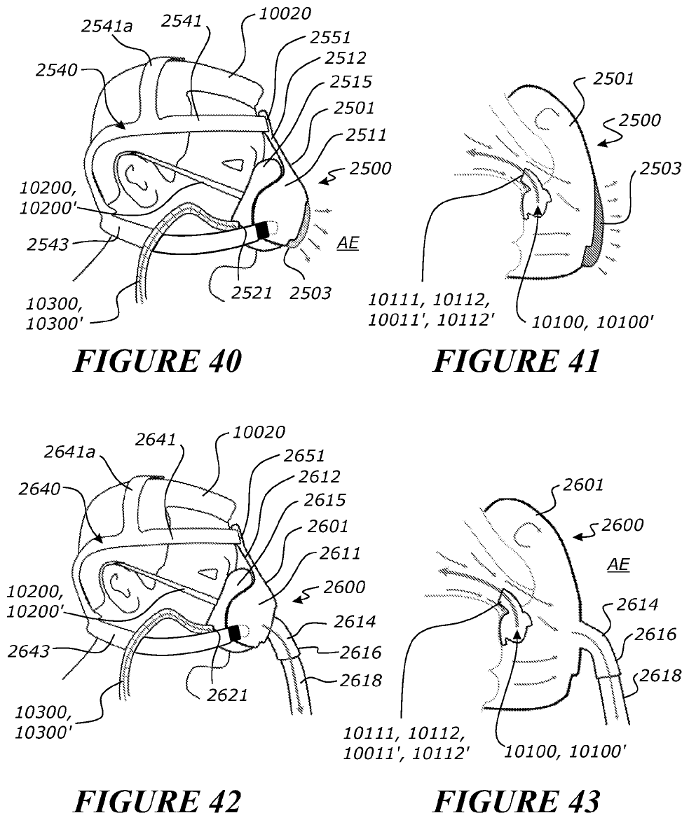

A twenty-fifth configuration mask 2500 is shown in FIGS. 40 and 41. The twenty-fifth configuration mask is a variant of the third configuration mask 300. Unless described as otherwise below, the features, functionality, and options are the same as the third configuration mask 300, and like reference numbers indicate like parts with the addition of 2200.

The mask body 2501 is configured to define a cavity or part thereof with the patient's face in use. The cavity is configured to at least accommodate a portion of the opening of the patient's airway.

In the configuration shown, the cavity is adapted to accommodate the patient's nose and mouth in use. In an alternative configuration, the cavity may be adapted to accommodate only the patient's nose in use, so as to enable access to and/or use of the mouth, e.g. for breathing, talking, eating etc and to enable unrestricted airflow to/from the patient's mouth.

In the configuration shown, the mask body 2501 comprises a frame 2511 and a cushion 2515 and is configured to define the cavity with the patient's face in use.

The cushion 2515 extends around an upper portion, lower portion, and side portions of the rear of the frame 2511.

The frame 2511 is substantially rigid.

The frame 2511 may be made from any of the materials described herein for other frames.

The cushion 2515 is configured to form a substantial seal against the patient's face.

The cushion 2515 may be made from any suitable compliant material, such as silicone for example.

The mask body 2501 comprises a filter 2503. The filter 2503 may have any of the features and functionality described herein for other filters.

The filter 2503 is arranged at a lower portion of the mask 2500 as shown. In the configuration shown, the filter 2503 is arranged to be proximal the patient's mouth in use to receive and filter a flow of fluid from the patient's mouth and/or nose.

The filter 2503 may be removable from the mask body 2501 and replaceable. Alternatively, the filter 2503 may be permanently attached to the mask body 2501.

In the configuration shown, the mask body 2501 does not have a transparent portion to permit a view of a portion of the patient's face in use. In an alternative configuration, the mask body 2501 can have such a transparent portion.

The mask 2500 comprises an interfacing feature 2521 configured to interface with the nasal cannula when donned on the patient. The interfacing feature may interact with the nasal cannula body, cannula tube or conduit, or any ancillary component of the cannula.

In the configuration shown, the interfacing feature 2521 comprises a recess or slot in the cushion 2515, wherein the recess or slot conforms to an external surface of the nasal cannula 10100, 10100′ such that the cushion substantially seals about the side arm(s) 10108, 10108′, 10109, 10109′ and/or patient conduit 10300, 10300′ of the nasal cannula.

The nasal cannula 10100, 10100′ and/or mask 2501 may comprise a mounting mechanism to engage the nasal cannula 10100, 10100′ with the mask 2501. For example, the mask 2501 may, comprise one or more fasteners mounted to the mask that are engageable with complementary features on the nasal cannula 10100, 10100′. The mounting mechanism may have any of the features and functionality described herein for other mounting mechanisms.

By providing a mounting mechanism and/or interfacing feature 2521 that positively engages the mask 2501 with the nasal cannula 10100, 10100′, usability may be improved and headgear requirements may be reduced.

The mask 2500 comprises a mask headgear 2540 or headgear connection features to secure the mask to the patient's face, which may be of any of the types of headgear or headgear connection features described herein.

In the configuration shown, the headgear 2540 comprises an upper headstrap 2541 having ends that connect to an upper portion of the mask body, and a lower headstrap 2543 having ends that connect to a lower portion of the mask body.

An upper portion 2512 of the frame 2511 extends upwardly from the mask body 2501 to a position corresponding to a patient's forehead in use. The upper headgear connection features or forehead support 2551 are provided at an upper end of the upper portion 2512 of the frame 2511. The upper headstrap 2541 can extend generally horizontally from the upper headgear connection features 2541 around the back of the patient's head.

The upper headstrap 2541 can have a crown strap 2541a to engage the top of the patient's head.

The lower headstrap 2543 can extend generally horizontally from the headgear connection features around the back of the patient's head or neck.

The upper headstrap 2541, lower headstrap 2543, and crownstrap 2541a can each be made up of one or more straps.

The upper headstrap 2541, lower headstrap 2543, and crownstrap 2541a may be elastic.

The upper headstrap 2541 and lower headstrap 2543 may be releasably connected to the frame 2511 and/or mask body 2501 via suitable connectors.

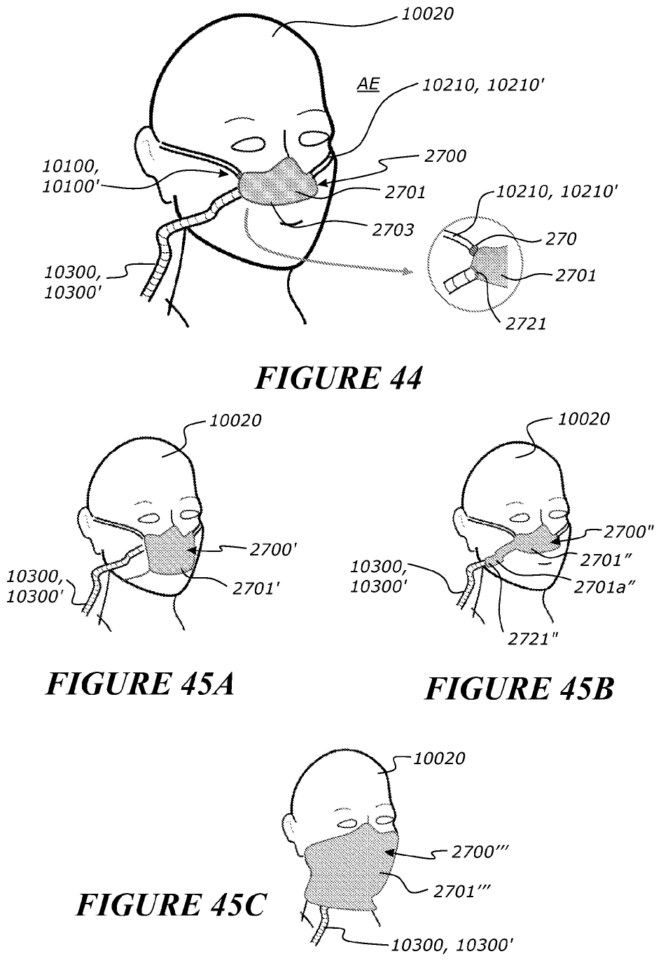

A twenty-seventh configuration mask 2700 is shown in FIG. 44. The twenty-seventh configuration mask is a variant of the ninth configuration mask 900. Unless described as otherwise below, the features, functionality, and options are the same as the ninth configuration mask 900, and like reference numbers indicate like parts with the addition of 1800.

The mask 2700 comprises a soft mask body 2701 that comprises a filter 2703 that is configured to filter a fluid from a patient facing side of the mask to a non-patient facing side of the mask which faces an ambient environment AE.

The mask body 2701 and/or filter 2703 comprises a flexible material that is configured to fit over and conform to the non-sealing nasal cannula 10100, 10100′.

In the configuration shown, the mask body 2701 is configured to fit over and conform to the non-sealing nasal cannula and the patient's nose.

The mask body 2701 and/or filter 2703 may be elastic or comprise an elastic material, to assist with conforming to the shape of the nasal cannula.

The mask body 2701 may be formed substantially entirely of the filter 2703. Alternatively, discrete part(s) of the mask body 2701 may be formed of the filter 2703, with other part(s) of the mask body 2701 formed of other material(s).

The mask 2701 comprises an interfacing feature 2721 configured to interface with the nasal cannula 10100, 10100′ when donned on the patient. The interfacing feature 2701 may interact with the cannula body, cannula tube or conduit, or any ancillary component of the cannula.

In the configuration shown, the interfacing feature 2721 comprises a recess or slot in the mask body 2701 that enables a portion of the nasal cannula 10100, 10100′ to extend therethrough.

The recess or slot conforms to an external surface of the nasal cannula 10100, 10100′ such that the mask body 2701 substantially seals about the portion of the nasal cannula such as the side arm(s) 10108, 10108′, 10109, 10109′ and/or patient conduit 10300, 10300′ of the nasal cannula.

The nasal cannula 10100, 10100′ and/or mask 2701 may comprise a mounting mechanism to engage the nasal cannula 10100, 10100′ with the mask 2501. For example, the mask 2701 may, for example, comprise one or more fasteners mounted to the mask that are engageable with complementary features on the nasal cannula 10100, 10100′.

In the configuration shown, the mask 2701 comprises a clip 2704 to clip onto headgear 10210, 10210′ of the nasal cannula 10100, 10100′.

FIGS. 45A-45C show alternative configurations of the twenty-seventh configuration mask. The alternative configuration masks 2701′, 2701″, 2701′″ may have of the features and functionality described for the twenty-seventh configuration mask.

The mask body 2701′ of the alternative configuration mask 2700′ of FIG. 45A is configured to fit over and conform to the non-sealing nasal cannula 10100, 10100′ as well as cover the patient's nose and mouth.

The mask body 2701″ of the alternative configuration mask 2700″ of FIG. 45B is configured to fit over and conform to the non-sealing nasal cannula 10100, 10100′ and the patient's nose. In an alternative configuration, the mask 2700″ may also cover the patient's mouth.

A portion 2701a″ of the mask body 2701″ is configured to extend around and/or be coextensive with the patient conduit 10300, 10300′ of the nasal cannula. The portion 2701a″ may wrap around the patient conduit 10300, 10300′ or may comprise an elastic sleeve portion that can be stretched over the nasal cannula and then contract onto the patient conduit 10300, 10300′. The portion 2701a″ provides the interfacing feature 2721″.

The mask body 2701′″ of the alternative configuration mask 2700′″ of FIG. 45C is configured to fit over and conform to the non-sealing nasal cannula 10100, 10100′ as well as cover the patient's nose and mouth. The mask body extends down toward the patient's chest from the patient's mouth.

The mask body 2701′″ may be in the form of a sleeve that extends around the back of the patient's head, or may be in the form of a sheet that hangs over the patient's face in use.

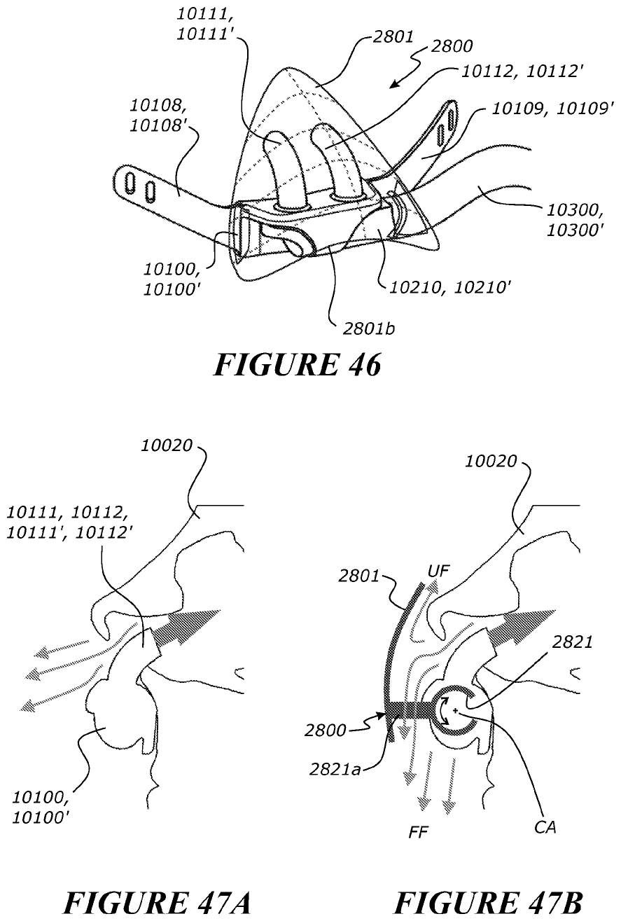

FIGS. 46 and 47B show a twenty-eighth configuration mask 2800.

The mask 2800 is configured to substantially surround an opening of a patient's airway. The mask 2800 has a mask body 2801 having a preformed shape that is configured to deflect fluids from the opening of a patient's airway in an intended direction.

The mask 2800 can shield and deflect and optionally filter droplets that are expelled from the opening of the patient's airway.

The mask can, for example, direct exhausted fluids which may include contaminants (includes pathogens) in a direction away from a medical professional and/or other persons in an ambient environment.

The mask 2800 may comprise an interfacing feature 2821 that is configured to, in use, interface with a patient interface provided on the patient. Alternatively, the mask 2800 may be integrally formed with the patient interface.

The patient interface may comprise a non-sealing patient interface.

The patient interface may comprise a nasal cannula.

In the configuration shown, the patient interface 10100, 10100′ comprises a non-sealing nasal cannula comprising one or more prongs 10111, 10111′, 10112, 10112′ that extend(s) into the patient's nasal passage(s) in use, side arms 10108, 10108′, 10109, 10109′ that extend laterally along the patient's face in use, and a patient conduit 10300, 10300′ adapted to connect to a gases source for delivering a gases flow to the patient via the prong(s).

In the configuration shown, the interfacing feature 2821 comprises a clip that is configured to couple to a manifold 10210, 10210′ and/or patient conduit 10300, 10300′ of the nasal cannula.

The clip 2821 comprises a rearwardly-open generally C-shaped clip that extends towards the patient interface and patient's face in use. The clip could have any suitable shape depending on the shape of the manifold and/or patient conduit.

The clip may be configured so that the mask 2800 is rotatable relative to the nasal cannula about a transverse axis CA through a length of a body of the nasal cannula (FIG. 47B). This enables the mask to be tilted to a desired orientation relative to the patient's face, depending on the shape and size of the face features and a desired deflection direction.

The desired orientation of the mask may depend on the position of the patient relative to the ambient environment. The mask would deflect flow in a preferential manner, not always directly down, and minimise flow in an undesirable direction. The preferential direction may avoid directing flows towards eyes or mouth of a patient.

The clip 2821 may be mounted directly to a rear surface of the mask body 2801. Alternatively, and as shown, an arm 2821a may extend rearwardly from the mask body 2801. The clip 2821 is mounted to an end of the arm 2821a that is distal from the mask body 2801. The arm 2821a spaces the mask body 2801 forward of the nasal cannula in use.

The mask body 2801 comprises a domed shape in at least one plane. In the form shown, the mask body comprises a domed shape in a vertical plane and in a horizontal plane.

The mask body 2801 comprises a concave shape when viewed from a patient-facing side of the mask, which is a convex shape when viewed from an ambient environment-facing side of the mask, in at least one cross-section.

The mask body 2801 comprises a generally triangular shape when viewed from a front of the mask. The base 2801b of the generally triangular shape may be recessed as shown to space the base 2801b further from the patient's face than would otherwise be the case with a straight base. That encourages flow of fluids downwardly from the bottom of the mask 2800.

The mask body 2801 could have any other suitable shape, such as a quadrilateral shape for example.

The mask body 2801 comprises a concave shape when viewed from a rear of the mask.

The edges of the mask body are smooth and/or corners of the mask body are rounded, to provide patient comfort.

In some configurations, the mask body 2801 may comprise a filter and/or absorbent material. The functionality, features, and options for the filter and absorbent material are as described herein for other filters and absorbent material.

For example, an interior of the mask body 2801 may comprise an absorbent material to encourage capture of any aerosols that contact the absorbent material.

In some configurations, the mask body 2801 is rigid. The mask body 2801 may have a thickness of about 1 mm or less.

In some configurations, the mask comprises injection moulded plastic such as polycarbonate, polypropylene, or acrylonitrile butadiene styrene (ABS). The plastic material may be transparent to provide a window that is advantageously configured to enable a medical professional to check that the nasal cannula 10100, 10100′ is in place in the patient's nares while using the mask 2800.

In alternative configurations, the mask body is inflatable.

In an alternative configuration, the mask body may comprise a solid frame with a shell of porous material with absorbent and/or filtering properties. In this configuration, the mask may both deflect a flow of fluids as well as filter and/or absorb fluids.

The mask 2800 has a light weight to enable it to easily be supported by the nasal cannula without causing discomfort.

The mask 2800 is configured to be lighter than the patient interface owing to material selection and/or structural design. For example, the mask 2800 may be up to about 50% of the weight of the patient interface, optionally up to about 30% of the weight of the patient interface, optionally up to about 10% of the weight of the patient interface.

In some configurations, the mask body 2801 comprises thin 0.3 mm-0.5 mm polymer sections.

The mask is adapted to direct fluids towards the patient's chest.

In the configuration shown, the mask 2800 is adapted to cover the nose of the patient. In an alternative configuration, the mask body 2801 may be larger so that the mask 2800 is adapted to cover the nose and mouth of the patient.

In the configuration shown, the upper portion of the mask body extends to a position adjacent and above the nasal tip of the patient's nose.

The upper portion of the mask body above the clip 2821 (and arm 2821a if provided) is longer than the lower portion of the mask body below the clip 2821 (and arm 2821a if provided), to encourage fluid flow FF from the bottom of the mask body 2801 and discourage upward fluid flow UF from the top of the mask body 2801.

Comparing FIGS. 47A and 47B shows that the mask 2800 directs exhausted fluids which may include contaminants (includes pathogens) in a direction away from a medical professional and/or other persons in an ambient environment, whereas flow from a non-sealing nasal cannula 10100, 10100′ without a mask 2800 may enable the flow to travel outwardly into the ambient environment.

In an alternative configuration of the mask 2800, a sealing cushion (not shown) may be provided on an upper portion of the mask body 2801 nasal tip to inhibit upward fluid flow from an upper surface of the mask body 2801.

In the configuration shown, the mask body 2801 comprises a one-piece preformed shaped component.

In an alternative configuration, the mask body 2801 may comprise a plurality of body portions that function together to deflect the fluid. The body portions may be angled differently from each other. For example, the mask body 2801 may comprise a left side portion and a right side portion that are angled towards each other to deflect fluids forward and down or that are angled away from each other to deflect fluids outward and down.

Any of the masks described herein may be provided in combination with a non-sealing patient interface configured to deliver a gases flow to a patient's airway, such as non-sealing nasal cannula 10100, 10100′ or any other suitable non-sealing patient interface, to provide an interface system.

The mask bodies each comprise a patient facing side and a non-patient facing side, the patient facing side configured to form an interior space with the patient's face when the mask is in contact with the patient. The interior space is configured to accommodate a portion of the non-sealing patient interface in use.

When the masks 1900-2400 are provided in an interface system, the fabric of the mask is configured to accommodate a portion of the non-sealing patient interface. For the masks 1900, 2200, and 2300, the fabric of the mask is configured to drape over a portion of the non-sealing patient interface.

When the mask comprises a mask headgear, the cannula headgear 10200, 10200′ and mask headgear are separate such that the mask is selectively securable to or removable from the patient independently of the non-sealing nasal cannula 10100, 10100′.

In the interface system, gases flow to the patient is only provided by the non-sealing nasal cannula 10100, 10100′ or other non-sealing patient interface. The mask does not comprise a gases supply or gases removal conduit and/or does not comprise a connection port for connecting to a gas source or gas removal device and/or is not adapted to actively deliver a flow of gases to the patient and/or is not adapted to actively remove a flow of gases from the patient.

However, in an alternative configuration, the mask may comprise a gases removal port or conduit to transmit fluids from the patient facing side of the mask to a remote filter or gases removal device. An example of such a configuration is the twenty-sixth configuration mask 2600 is shown in FIGS. 42 and 43. The twenty-sixth configuration mask is a variant of the twenty-fifth configuration mask 2500. Unless described as otherwise below, the features, functionality, and options are the same as the twenty-fifth configuration mask 2500, and like reference numbers indicate like parts with the addition of 100.

In this configuration, the mask body 2601 comprises a gases removal port 2614.

The gases removal port 2614 is provided in an expiratory limb 2616.

The expiratory limb 2616 is connected to or connectable to a gases removal conduit 2618.

The gases removal conduit may be in fluid communication with a remote filter and/or with a gases removal device such as a suction unit and/or extraction vent for example. The mask 2600 can therefore be used to filter a fluid from a patient facing side of the mask via a downstream filter, and/or to actively remove a flow of gases from the patient.

In some configurations, the mask body 2601 of the mask may comprise a filter and/or an absorbent material as described herein.

The gases flow provided by the nasal cannula may be provided at high flow rates, optionally up to about 120 L/min, or about 10-70 L/min. The gases flow provided by the cannula 10100, 100100′ may be humidified.

Any of the masks disclosed herein may comprise the absorbent material, and may comprise a transmission arrangement configured to transfer the liquid absorbed by the absorbent material away from the absorbent material. The transfer arrangement may be configured to transfer the absorbed liquid to an absorbent pad.

The masks may be considered a first patient interface and the non-sealing patient interface may be considered a second patient interface. Any of the masks disclosed herein may comprise one or more interfacing features configured to, in use, interface with the second patient interface provided on the patient. The interfacing feature(s) could be any of the types disclosed herein.

The masks are disclosed herein as being used in combination with non-sealing nasal cannulas 10100, 10100′. However, the masks could be used in combination with any other suitable types of non-sealing patient interfaces, such as any non-sealing oral and/or patient interface for example.

In the interface systems, the interfacing feature (when provided) may comprise an aperture provided in the mask body and/or between the mask body and the second retention mechanism (e.g. mask headgear) to allow a portion of the nasal cannula to extend through, optionally to allow the patient conduit of the nasal cannula to extend through.

Any of the masks disclosed herein could comprise a vent to provide fluid communication between the patient facing side of the mask and the ambient environment. The vent may comprise the filter or a portion thereof.

The vent may be configured to allow for a certain flowrate of a gases flow through it. For example, the vent may be configured to allow for a flow rate of gases flow through it of up to 450 L/min, optionally of about 10-450 L/min. The vent may be adjustable.

Any of the masks disclosed herein could be configured for us with a patient interface that comprises one or more venting apertures. Any of the masks disclosed herein could be configured to filter and/or deflect fluids that exit the patient interface via venting aperture(s). Any of the masks disclosed herein could be configured to be positioned proximate the venting aperture(s) in a patient interface to filter and/or deflect fluids exiting the venting aperture(s).

One or more of the interfacing features or mounting mechanisms described herein could be used to mount the mask over the venting aperture(s) in the patient interface. The mask can cover the venting aperture(s) and filter and/or deflect fluids that exit the patient interface via the venting aperture(s).

The mask may be a non-sealing mask.

For example, the mask may have a configuration similar to the twenty-eighth configuration mask of FIGS. 46 and 47B. The mask can shield and deflect and optionally filter droplets that are emitted through the vent holes.

The mask 2801 may optionally have an absorbent material and/or liquid reservoir of any of the types described herein to capture and hold the captured liquid.

The mask body 2801 can block the line of sight emission of droplets. However, because the mask body 2801 acts as a non-sealing shield blocking the droplets, the risk of fully occluding the venting gases path and adversely affecting the functionality of the patient interface is negligible.

Although the present disclosure has been described in terms of certain embodiments, other embodiments apparent to those of ordinary skill in the art also are within the scope of this disclosure. Thus, various changes and modifications may be made without departing from the spirit and scope of the disclosure. For instance, various components may be repositioned as desired. Features from any of the described embodiments may be combined with each other and/or an apparatus may comprise one, more, or all of the features of the above described embodiments. Moreover, not all of the features, aspects and advantages are necessarily required to practice the present disclosure. Accordingly, the scope of the present disclosure is intended to be defined only by the claims that follow.

1-89. (canceled)

90. A mask configured to surround an opening of a patient's airway, the mask comprising:
a mask body comprising a removable filter configured to filter a fluid from a patient facing side of the mask to a non-patient facing side of the mask, wherein the non-patient facing side faces an ambient environment; and
an interfacing feature configured to interface with a patient interface provided on a patient.

91. The mask of claim 90, wherein the removable filter comprises a material that is permeable to fluid flow.

92. The mask of claim 90, wherein the removable filter comprises a material that is substantially impermeable to bulk flow of liquid water.

93. The mask of claim 90, further comprising an absorbent material to absorb a liquid.

94. The mask of claim 90, wherein the patient interface is non-sealing.

95. The mask of claim 94, wherein the patient interface is a non-sealing nasal cannula comprising at least one prong that is configured to extend into a patient's nasal passage, a pair of side arms that is configured to extend laterally along a patient's face, and a patient conduit configured to connect to a gases source for delivering a gases flow to the patient via the at least one prong.

96. The mask of claim 95, wherein the mask is selectively securable to or removable from a face of the patient independently of the non-sealing nasal cannula.

97. The mask of claim 90, wherein the mask:
does not comprise a gases supply or gases removal conduit; or
does not comprise a connection port for connecting to a gas source or gas removal device; or
is not adapted to actively deliver a flow of gases to the patient; or
is not adapted to actively remove a flow of gases from the patient.

98. The mask of claim 90, wherein the mask comprises a gases removal port or conduit to transmit fluids from the patient facing side of the mask to a remote filter or gases removal device.

99. The mask of claim 90, wherein the mask comprises a frame, and wherein the mask body is integral with or releasably coupled to the frame

100. The mask of claim 95, wherein the mask comprises a cushion configured to seal about or against the patient's face, and wherein the interfacing feature comprises a recess or slot in the cushion, wherein the recess or slot conforms to an external surface of the non-sealing nasal cannula such that the cushion substantially seals about the side arms and/or the patient conduit of the non-sealing nasal cannula.

101. The mask of claim 95, wherein the interfacing feature comprises an aperture provided in the mask body to allow a portion of the non-sealing nasal cannula to extend through, optionally to allow the patient conduit of the non-sealing nasal cannula to extend through.

102. The mask of claim 95, wherein the mask comprises a frame shaped to conform to an external surface of the patient conduit of the non-sealing nasal cannula, and wherein the interfacing feature comprises a first mounting structure to releasably couple the frame to the patient conduit.

103. The mask of claim 95, wherein the interfacing feature comprises at least one mounting mechanism to couple the mask to the non-sealing nasal cannula.

104. The mask of claim 90, further comprising an attachment mechanism, wherein the attachment mechanism is releasably attached to a patient's face.

105. The mask of claim 90, wherein the patient interface comprises a non-sealing nasal cannula, wherein the mask body and/or filter comprises a flexible material configured to conform to the non-sealing nasal cannula.

106. An interface system comprising:
a non-sealing patient interface configured to deliver a gases flow to a patient's airway; and
a mask comprising:
a mask body comprising a patient facing side and a non-patient facing side, the patient facing side configured to form an interior space with a patient's face when the mask is in contact with a patient, the interior space configured to accommodate a portion of the non-sealing patient interface;
a filter configured to filter a fluid from a patient-facing side of the mask to a non-patient facing side of the mask, wherein the non-patient facing side faces an ambient environment; and
an interfacing feature configured to interface with the non-sealing patient interface.

107. The interface system of claim 106, wherein the non-sealing patient interface comprising a first retention mechanism configured to retain the non-sealing patient interface on the patient's face in use.

108. The interface system of claim 107, further comprising a second retention mechanism configured to retain the mask on a patient's face, wherein the first retention mechanism and the second retention mechanism are separate such that the mask is selectively securable to or removable from a patient independent of the non-sealing patient interface.

109. A mask configured to surround an opening of a patient's airway, the mask comprising:
a mask body having a preformed shape that is configured to deflect fluids from the opening of a patient's airway in an intended direction; and
wherein either the mask further comprises an interfacing feature configured to interface with a patient interface provided on a patient, or the mask is integrally formed with a patient interface.
